SEQUENCE LISTING (7 sequences)

SEQ ID NO: 1 - DNA, 25 bp, Artificial Sequence
  Description of Artificial Sequence  Synthetic primer
ccgccttgagcggcaggtttaaagg

SEQ ID NO: 2 - DNA, 24 bp, Artificial Sequence
  Description of Artificial Sequence  Synthetic primer
catgtcaccaagctcgacgaggtg

SEQ ID NO: 3 - DNA, 25 bp, Artificial Sequence
  Description of Artificial Sequence  Synthetic primer
ccgccttgagcggcaggtttaaagg

SEQ ID NO: 4 - DNA, 20 bp, Artificial Sequence
  Description of Artificial Sequence  Synthetic primer
tggatgtggaatgtgtgcga

SEQ ID NO: 5 - protein, 70 aa, Artificial Sequence
  Description of Artificial Sequence  Synthetic construct
EVIKHKLCRRGKCQKEAKRSSADKGVALSLDDVKRHNPCESLRGHPAGMTYAANILPHHPARGTFEDFTC

SEQ ID NO: 6 - protein, 22 aa, Artificial Sequence
  Description of Artificial Sequence  Synthetic peptide
MELKTEEEEVGGVQPVSIQAFA

SEQ ID NO: 7 - protein, 22 aa, Artificial Sequence
  Description of Artificial Sequence  Synthetic peptide
MELDEGDSPRDLVAFANSCTLH

(19) United States
(12) Patent Application Publication
(10) Pub. No.: US 2007/0087964 A1
(43) Pub. Date: Apr. 19, 2007

(54) NOVEL COMPOSITIONS AND METHODS FOR MODULATING THE ACID-SENSING ION CHANNEL (ASIC)

(75) Inventors: MICHAEL J. WELSH, Riverside, IA (US); JOHN A. WEMMIE, Iowa City, IA (US)

Correspondence Address: MCKEE, VOORHEES & SEASE, P.L.C., 801 GRAND AVENUE, SUITE 3200, DES MOINES, IA, 50309-2721, US

(73) Assignees: UNIVERSITY OF IOWA RESEARCH FOUNDATION, Iowa City, IA (US); DEPARTMENT OF VETERAN'S AFFAIRS, UNITED STATES, Washington, DC (US)

(21) Appl. No.: 11/464,268

(22) Filed: Aug. 14, 2006

Related U.S. Application Data
(63) Continuation of application No. 10/112,280, filed on Mar. 29, 2002, now abandoned.

(51) Int. Cl. A61K 38/17; A61K 31/498

(52) U.S. Cl. 514/12; 514/249

(57) ABSTRACT

Novel compositions for modulating acid-sensing ion channels (ASIC) function comprising ASICα, ASICβ, and BNC1 and derivatives thereof; methods for modulating ASIC function and methods for treating cognitive disorders and for memory enhancement using the novel compositions of the invention; and a method for increasing synaptic plasticity are described.

### CROSS-REFERENCE TO RELATED APPLICATIONS

This application is a continuation of U.S. patent application Ser. No. 10/112,280 filed on Mar. 29, 2002, the contents of which are hereby incorporated by reference in its entirety.

### GRANT REFERENCE

Work for this invention was funded in part by grants from Howard Hughes Medical Institute, Veteran's Administration Research Career Development Award (JAW), NINDS Grant No. NS 38890, NIH Grants GM 57654, HL 64645 and HL 14388. The United States government may have certain rights in this invention.

### FIELD OF THE INVENTION

This invention relates to acid-sensing ion channel (ASIC) agonists, antagonists and modulators. In particular, this invention relates to pharmaceutical compositions, dietary supplements and methods of treatment which modulate the acid-sensing ion channel (ASIC) for treatment of Central Nervous System (CNS) disorders such as seizures and strokes through synaptic plasticity, treatment of cognitive disorders, and for memory enhancement.

### BACKGROUND OF THE INVENTION

The present invention relates to pharmaceutical compositions for the treatment of strokes and seizures and improved synaptic plasticity for learning and memory capabilities. Further, the invention relates to a method of modulating the activity of the ASIC receptors in mammals through the use of an antagonist or agonist and their uses in the treatment of conditions associated with ASIC receptor activity.

It is known in the art that the N-methyl-D-aspartate (NMDA) receptor plays a major role in the synaptic plasticity which underlies many higher cognitive functions, such as memory and learning, as well as in certain nociceptive pathways and in the perception of pain (Collingridge et al., The NMDA receptor, Oxford University Press, 1994). In addition, certain properties of NMDA receptors suggest that they may be involved in the information-processing in the brain which underlies consciousness itself.

The NMDA receptor is a postsynaptic, ionotropic receptor which is responsive to, inter alia, the excitatory amino acids glutamate and glycine and the synthetic compound NMDA, hence the receptor name. The NMDA receptor controls the flow of both divalent (Ca⁺⁺) and monovalent (Na⁺, K⁺) ions into the postsynaptic neural cell through a receptor associated channel (Foster et al., “Taking apart NMDA receptors”, Nature, 329:395-396, 1987).

NMDA receptor antagonists are therapeutically valuable for a number of reasons, such as the following three specific reasons. Firstly, NMDA receptor antagonists confer profound analgesia, a highly desirable component of general anesthesia and sedation. Secondly, NMDA receptor antagonists are neuroprotective under many clinically relevant circumstances (including ischemia, brain trauma, neuropathic pain states, and certain types of convulsions). Thirdly, NMDA antagonists confer a valuable degree of amnesia.

However, it is clear from the prior art that there are a number of drawbacks associated with current NMDA receptor antagonists. These include the production of involuntary movements, stimulation of the sympathetic nervous system, induction of neurotoxicity at high doses (which is pertinent since NMDA receptor antagonists have low potencies as general anesthetics), depression of the myocardium, and proconvulsions in some epileptogenic paradigms e.g., “kindling” (Walz P et al., Eur. J. Neurosci. 1994; 6:1710-1719). In particular, there have been considerable difficulties in developing new NMDA receptor antagonists that are able to cross the blood-brain barrier. This factor has also limited the therapeutic applications of many known NMDA antagonists. None of the foregoing explanations or discoveries has found a satisfactory mechanism for modulating the NMDA receptor function. The present invention thus seeks to provide a more safe and improved ASIC receptor antagonist for general pharmaceutical use to treat seizures, strokes and other conditions associated with acidosis and high extracellular glutamate. In addition, ASIC receptor agonists will allow treatment and preventative uses for conditions associated with impaired learning and memory.

The present invention relates to pharmaceutical compositions in the prevention and treatment of CNS disorders which have been attributed to neurotransmitter system dysfunction. CNS disorders are a type of neurological disorder. CNS disorders can be drug induced; can be attributed to genetic predisposition, infection or trauma; or can be of unknown etiology. CNS disorders comprise neuropsychiatric disorders, neurological diseases and mental illnesses; and include neurodegenerative diseases, behavioral disorders, cognitive disorders and cognitive affective disorders. There are several CNS disorders whose clinical manifestations have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors). Several CNS disorders can be attributed to a cholinergic deficiency, a dopaminergic deficiency, an adrenergic deficiency and/or a serotonergic deficiency. CNS disorders of relatively common occurrence includes presenile dementia (early onset Alzheimer's disease), senile dementia (dementia of the Alzheimer's type, Parkinsonism including Parkinson's disease, Huntington's chorea, tardive dyskinesia, hyperkinesia, mania, attention deficit disorder, anxiety, dyslexia, schizophrenia and Tourette's syndrome.

The treatment and prevention of strokes are just one of the conditions of the CNS that ASIC antagonists can assist with through modulation of the acid-sensing ion channel. A stroke has the same relationship to the brain as a heart attack does to the heart; both result from a blockage in a blood vessel that interrupts the supply of oxygen to cells, thus killing them. Blood is supplied to the brain through two main arterial systems: the carotid arteries that come up through the front of the neck and the vertebral arteries that come up through the rear of the neck. Brain cells require a constant supply of oxygen to stay healthy and function properly. The brain receives about 25% of the body's oxygen supply, but it cannot store oxygen; a reduction of blood flow for even a short period of time can. be disastrous. The consequences of a stroke, the type of functions affected and the severity, depend on where in the brain the blockage has occurred and on the extent of the damage.

The brain area affected determines the neurological effects of a stroke. One of the most common types of stroke is blockage of one of the middle cerebral arteries that supplies the midportion of one brain hemisphere. For instance, if the middle cerebral artery is blocked on the left side of the brain, the person is likely to become almost totally demented because of lost function in Wernicke's speech comprehension area; he or she also becomes unable to speak words because of loss of Broca's motor area for word formation. In addition, lost function in other neural motor control areas of the left hemisphere can create spastic paralysis of all or most muscles on the opposite side of the body.

In a similar manner, blockage of a posterior cerebral artery will cause infarction of the occipital pole of the hemisphere on the same side and loss of vision in both eyes in the half of the retina on the same side as the stroke lesion. Especially devastating are strokes that involve the blood supply to the hindbrain and midbrain because they can block conduction in major pathways between the brain and spinal cord, causing totally incapacitating sensory and motor abnormalities.

During brain ischemia caused by stroke or traumatic injury, excessive amounts of the excitatory amino acid glutamate are released from damaged or oxygen deprived neurons. This excess glutamate binds to the NMDA receptor which opens the ligand-gated ion channel thereby allowing Ca⁺⁺ influx producing a high level of intracellular Ca⁺⁺ which activates biochemical cascades resulting in protein, DNA and membrane degradation leading to cell death. This phenomenon, known as excitotoxicity, is also thought to be responsible for the neurological damage associated with other disorders ranging from hypoglycemia and cardiac arrest to epilepsy. In addition, there are preliminary reports indicating similar involvement in the chronic neurodegeneration of Huntington's, Parkinson's and Alzheimer's diseases.

The treatment and prevention of seizures of the CNS is also improved with ASIC antagonists. Epilepsy is not a single disorder, but covers a wide spectrum of problems characterized by unprovoked, recurring seizures that disrupt normal neurologic functions. Epileptic seizures occur when a group of neurons in the brain become activated simultaneously, emitting sudden and excessive bursts of electrical energy. This hyperactivity of neurons can occur in various locations in the brain and, depending on the location, have a wide range of effects on the sufferer, from brief moments of confusion to minor spasms to loss of consciousness. The nerves themselves may be damaged or problems might occur in the neurotransmitters. The neurotransmitter, gamma amniobutyric acid (GABA) seems to be particularly important in suppressing seizures. Experiments also suggest that deficiencies in a receptor of the neurotransmitter serotonin may help promote epileptic seizures. Epilepsy falls into two main categories: partial, or focal, seizures and generalized seizures. Within these two categories are a number of subtypes, each of which requires different therapeutic approaches, so an accurate diagnosis is important. In addition, some cases of epilepsy can be a hybrid of subtypes, while others defy precise categorization. Nonetheless, elimination of ASIC activity has been found to block the damaging effects that occur during seizures.

There are many memory-related conditions for which therapeutic treatments are under investigation, such as methods to enhance memory or to treat memory dysfunction. For example, memory dysfunction is linked to the aging process, as well as to neurodegenerative diseases such as Alzheimer's disease. In addition, memory impairment can follow head trauma or multi-infarct dementia. Many compounds and treatments have been investigated which can enhance cognitive processes, that is, which can improve memory and retention. In the present invention, the ASIC receptor enhances learning and memory.

This invention describes the inactivation of the acid-sensing ion channel whereby the ASIC dampens excitatory synaptic transmission, which has been implicated in the pathophysiology of seizures and strokes and impairs learning and memory. In addition, this invention identifies that pharmacological agents that block (antagonists) ASIC can inhibit the damaging effects of acidosis and excess glutamate release, which occur during seizures and strokes. The present invention also describes how pharmacological agents that activate (agonists) ASIC can enhance learning and memory. The results of the present invention resemble those of “knocking out” the NMDA receptor but without the severe side effects. Therefore, drugs acting on the ASIC receptor therefore are expected to have an enormous therapeutic potential. Especially due to the fact that the severe side effects of the now used NMDA receptor are not present when ASIC receptor disruption is utilized.

For the foregoing reasons, there is a need for determination, characterization and application of ASIC modulation of synaptic plasticity involved in seizures and strokes and excitatory synaptic transmission as a method of treatment for learning and memory loss.

Accordingly, a primary objective of the invention is pharmaceutical compositions for the treatment and prevention of strokes, seizures and loss of memory using ASIC antagonists or agonists, respectively.

Another objective of the invention is a dietary supplement to treat and prevent CNS disorders.

A further objective of the invention is a method to disrupt ASIC thereby affecting synaptic plasticity that directly effects seizures and strokes.

A further objective of the invention is a method to enhance memory and learning activating ASIC or utilizing pharmacological agents.

Yet another objective of the invention is a method for screening compositions to identify ASIC.

The method and means of accomplishing each of the above objectives will become apparent from the detailed description of the invention which follows. Additional objectives and advantages of the invention will be set forth in part in the description that follows, and in part will be obvious from the examples, or may be learned by the practice of the invention. The objectives and advantages of the invention will be obtained by means of the instrumentalities and combinations, particularly pointed out in the claims of the invention.

### SUMMARY OF THE INVENTION

The present invention identifies that newly discovered ASIC antagonists can block the damaging effects of acidosis and high extracellular glutamate, in conditions such as strokes and seizures, without the severe side effects seen with NMDA antagonists. In addition, ASIC agonists can enhance memory and learning.

Based on this finding, pharmacological agents that can activate or block ASIC will have less severe side effects and will be better tolerated treatments for neurologic damage that results from stroke, seizures and for memory loss. The present invention further identifies the function of acid-gated currents in general and H+-gated DEG/ENaC channels that potentiates the effects of acid-sensing ion channels molecular identity and physiologic function which has remained unknown until now thereby allowing for new treatments and methods for CNS disorders.

### BRIEF DESCRIPTION OF THE DRAWINGS

FIGS. 1A-1C are graphs and blot analyses demonstrating the generation of ASIC knockout mice. (A) Strategy for targeted disruption of the ASIC gene locus. Shown above is schematic of anticipated topology of ASIC protein (N, amino-terminus; C, carboxyl-terminus; TM, transmembrane domain; ECD, extracellular domain; stippled region is coded by targeted exon; arrowhead, spice junction). Also shown are wild-type genomic locus, targeting vector, and targeted locus. (B) Southern blot analysis of Sac I digested genomic DNA from liver of animals with indicated genotype and hybridized to a probe outside of the targeting vector (probe A) or to a probe corresponding to the deleted exon (probe B). (C) Northern blot analysis of total brain RNA hybridized to a probe for ASICα or BNC1. Equivalent loading of RNA was verified by ethidium bromide (ETBr) staining of ribosomal RNA. FIGS. D and E demonstrate Nissl staining of 5 μm coronal sections through the hippocampus and cerebellar cortex, respectively. FIGS. F and G demonstrates immunoprecipitation of whole brain extracts. (F) demonstrates immunoprecipitation of whole brain extracts with anti-ASICαβ anti-sera and western blotted with the antibodies indicated on the left. Equivalent amounts of total protein from −/− and +/+ mice were used as starting material. As a positive control for ASICα and ASICβ, protein extracts were used from COS cells transfected with the respective cDNAs. Non-transfected COS cells yield no signal when probed with anti-ASIC antibodies (not shown). (G) Immunoprecipitation and western blotting with anti-ASICαβ of protein extracts from dissected hippocampus.

FIG. 2 demonstrates the co-distribution of PSD-95 and ASIC in transfected rat hippocampal neurons. (A) ASIC-FLAG immunofluorescence. (B) PSD-95 GFP fluorescence. Arrowhead indicates axon. Side by side comparison of signal from identical regions of the neuron indicated by A1, B1 and A2, B2 show foci of co-distribution of PSD-95 and ASIC (arrowheads).

FIG. 3 shows ASIC enriched in synaptosome-containing brain fractions. Western blotting with antibodies to ASIC, PSD-95 and GluR2/3 indicated on left. H, crude brain homogenate; SF, synaptosome-containing fraction.

FIG. 4 demonstrates how transient acid-evoked cation currents are absent in hippocampal neurons from ASIC knockout mice. (A) Representative whole cell recordings of pyramidal neurons from +/+ and −/− mice in response to application of agonist by bar: GABA, 200 μM; AMPA, 200 μM; NMDA, 200 μM. (B) Bar graph of average peak currents elicited by pH 5, GABA, AMPA, and NMDA. Error bars represent SEM. Asterisk indicates p<0.00001. Differences in response of +/+ and −/− neurons to GABA, AMPA, and NMDA were not statistically significant (+/+, n=32; −/−, n=41).

FIGS. 5A-5E demonstrates baseline synaptic transmission is normal and LTP is impaired in hippocampal slices from ASIC knockout mice. (A) EPSP amplitude plotted as a function of stimulus intensity shows no significant difference between slices from +/+ and −/− mice. (B) Analysis of components of baseline EPSP sensitive to the non-specific ionotropic glutamate receptor antagonist kynurenic acid (KA), the NMDA receptor antagonist D-2-amino-5-phosphopentanoic acid (D-APV), and the AMPA receptor antagonist 6-cyano-7-nitroquinoxaline-2,3-dione (CNQX). Left column, KA (5 mM) abolished EPSPs in slices from +/+ (n=3) and −/− (n=3) mice. Middle column, D-APV (50-100 μM) did not significantly change the EPSPs from either +/+ (n=8) or −/− (n=4) mice under the conditions used for the LTP experiments (1.3 mM Mg²⁺). Right column, EPSPs from +/+ (n=3) and −/− (n=3) mice were recorded in the presence of (1) 1.3 mM Mg²⁺, (2) low Mg²⁺(0.1 mM), 10 μM CNQX, or (3) low Mg²⁺(0.1 mM), 10 μM CNQX, and D-APV (50 μM). The non-CNQX sensitive component of the EPSP was not different between groups and in both groups the EPSP was blocked by CNQX plus D-APV. (C) LTP is impaired in −/− slices. Average normalized EPSP slope plotted vs. time. A1, A2, B 1, B2-representive tracings at indicated times; HFS, application of 100 Hz. for 1 s. (+/+, n=8; −/−, n=13). Forty min. after HFS the average FEPSP slope was 99±5% of pre-HFS values in −/− mice and 184±7% of pre-HFS values in +/+ mice, p=0.000005. (D) LTP is rescued in −/− slices in the presence of low Mg²⁺(0.1 mM, bar) (+/+, n=6; −/−, n=6). Mean EPSP values 40 min. after HFS in +/+ and −/− mice were 152±5% and 156±8% baseline respectively (p=0.99). As expected, a reduction in Mg²⁺ concentration caused a slight increase in baseline EPSP slope in both groups of mice. To maintain comparable baseline transmission, the stimulus intensity was reduced slightly in both groups 15 min prior to HFS (downward arrow). (E) Application of phorbol 12-myristate 13-acetate (10 μM PMA, bar) restores LTP in −/− slices (+/+, n=6; −/−, n=5). Mean EPSP amplitudes at 40 min. following HFS were not different (−/−, 158±11%; +/+, 167±15%; p=0.41). When PKC was activated in the brain slice by the addition of PMA, baseline EPSP amplitude increased slightly in 2 of 6 slices from the −/− group and 1 of 5 slices from +/+ group. Increases in baseline EPSP were corrected by decreasing stimulus intensity (downward arrow). A stable EPSP baseline was observed for 15 min. before HFS.

FIGS. 6A-6G are graphs illustrating EPSP facilitation during HFS is impaired but paired pulsed facilitation (PPF) is intact in ASIC −/− mice. (A) Averaged responses of the first 10 EPSPs during HFS from +/+ mice (n=8). (B) Averaged responses of the first 10 EPSPs during HFS from ASIC −/− mice (n=8). (C) Superimposed normalized responses to HFS from +/+ (thin tracing) and −/− mice (thick tracing). All the amplitudes of EPSP during HFS were normalized to the amplitude of the first EPSP in each slice. (D) Amplitude of 2^{nd}, 5^{th}, 10^{th}, 20^{th} EPSPs normalized to the amplitude of the first EPSP. The 2^{nd}, 5^{th}, 10^{th} EPSPs are significantly different between +/+ and −/− mice (***: p<0.001, **: p<0.05). (E) Averaged responses of the first 10 EPSPs during HFS from wild-type mice in the presence of D-APV (50 μM) shows a remarkable resemblance to ASIC −/− slices. (F) Representative traces of paired pulse facilitation in +/+ and −/− mice at 20 and 50 ms intervals. (G) Averaged PPF ratio of +/+ (n=21) and −/− mice (n=22) at 20 and 50 ms intervals. There was no significant difference in PPF between +/+ and −/− mice; 20 ms (p=0.81), 50 ms (p=0.93).

FIGS. 7A-7F illustrates results from the Morris water maze showing how a mild deficit in spatial memory in ASIC null mice can be overcome by intensive training. (A) Escape latency during training, 1 trial per day for 11 days. Regression analysis of learning curves of two groups revealed a significant difference in slope (t(131)=2.93; p<0.004; +/+, n=10; −/−, n=9). Repeated measures analysis of variance with all 11 trials revealed a difference that was not within the standard confidence interval ((F1,17)=3.20; p<0.095), although analysis of variance of last five trials revealed a significant effect of group factor (F(1,17)=5.43; p<0.035). Due to the difference in learning curve slope the difference in learning proficiency is more apparent with later trials. (B) Probe trial. Percent time spent in indicated quadrant; training, T; adjacent left, L; adjacent right, R; opposite, O.

Within the +/+ group, analysis of differences of least squares means revealed a significant difference between training quadrant and the other three quadrants (t(36)>2.9, p<0.006; indicated by asterisk). Within the −/− group, the differences between training quadrant and the other three quadrants were not stastically significant (t(32)<1.6, p>0.11). (C) Platform crossings during probe trial. Within the +/+ group, analysis of differences of least squares means revealed a significant difference between training quadrant and quadrants L and O (t(36)>2.1, p<0.04, indicated by asterisk.) The difference between T and R was not as pronounced (t(36)=1.98, p=0.055). Within the −/− group the differences between training quadrant and the other three quadrants were not statistically significant (t(32)<0.73, p>0.47). No significant difference was observed between groups. (D) Escape latency during platform reversal test when platform was placed in training quadrant, T, or opposite quadrant, O. Analysis by paired t-test revealed a significant difference between quadrant T and O for the +/+ mice (t(9)=5.4; p<0.0001, indicated by asterisk), but not for the −/− mice (t(8)=1.45; p=0.19.) The difference between groups was not statically significant. (E) The performance of +/+ and −/− mice is the same during more intensive training, 3 blocks of 4 trials per day for 3 days. Repeated measures analysis of variance revealed no statistical difference between groups. (F) (1,12)=0.045; p=0.83; +/+, n=7; −/−, n=7). The difference between groups during once a day training is lost with more intensive training. All error bars represent SEM.

FIGS. 8A-8B demonstrate how eyeblink conditioning is substantially impaired and rotarod performance is normal in ASIC knockout mice. (A) Percentage of conditioned responses during indicated session of 100 trials per day. An analysis of variance revealed a significant interaction of the group (+/+ vs. −/−) and condition (Paired vs. Unpaired) factors, F (1,19)=4.657, p<0.05. Post-hoc tests (Tukey HSD) revealed a significantly greater difference between paired and unpaired groups in the +/+ mice (p<0.05), but not in the −/− mice. The results indicate that the +/+ mice developed greater associative eyeblink conditioning relative to the −/− mice. (B) The performance of +/+ and −/− mice is similar on the accelerating rotarod 0.3 rpm/s. Mice received three trials per day. Initial speed was 3 rpm. Averaged maximum rpm achieved before falling is plotted vs. the day of the trials (+/+, n=17; −/−, n=19).

### DETAILED DESCRIPTION OF PREFERRED EMBODIMENTS

Acid-sensing ion channels (ASICs) are members of the DEG/ENaC superfamily of Na⁺ permeable channels, which includes the FMRFamide-gated channel (FaNaCh). They are activated by a drop of pH below 6.8 and desensitize rapidly which has raised the question of their functional role (Akaike et al., 1994). The current invention utilizes the finding that ASIC contributes to synaptic plasticity, learning and memory in such a way as to provide useful compositions and pharmaceutical agents which can aid regulation of these physiological responses.

Acid-activated cation currents have been detected in central and peripheral neurons for more than 20 years (Gruol et al., 1980; Krishtal and Pidoplichko, 1981). In the central nervous system, they have been observed in the hippocampus (Vyldicky et al., 1990), cerebellum (Escoubas et al., 2000), cortex (Varming, 1999), superior colliculus (Grantyn and Lux, 1988), hypothalamus (Ueno et al., 1992), and spinal cord (Gruol et al., 1980).

Currents evoked by a fall in extracellular pH vary in pH sensitivity, with half maximal stimulation ranging from pH 6.8 to 5.6 (Varming, 1999). Despite the wide spread distribution of H⁺-gated currents in the brain, neither their molecular identity nor their physiologic functions are known.

Although many central neurons possess large acid-activated currents, their molecular identity and physiologic function have remained unknown. Previous to the discovery of ASIC receptors, the NMDA receptor has been implicated during development in specifying neuronal architecture and synaptic connectivity and may be involved in experience dependent synaptic modifications. NMDA receptors are also thought to be involved in long term potentiation, Central Nervous System (CNS) plasticity, cognitive processes, memory acquisition, retention, and learning. However, activation of the NMDA receptor, which occurs only under conditions of coincident presynaptic activity and postsynaptic depolarization, has displayed significant difficulty. Current medications that are prescribed to either activate or block the NMDA receptor and influence glutamatergic synaptic transmission are poorly tolerated because of severe side effects.

Recently researchers identified a family of cation channels that are gated by reductions in pH. These proteins, called ASICs, are related to amiloride-sensitive epithelial sodium channels (ENaCs) and the degenerin/mec family of ion channels from Caenorhabditis elegans (Waldmann et al., 1997). The acid-sensing DEG/ENaC channels respond to protons and generate a voltage-insensitive cation current when the extracellular solution is acidified. This invention found the acid-sensing ion channel (ASIC) to be present in the hippocampus, enriched in synaptosomes, and localized at dendritic synapses in hippocampal neurons. Disruption of the ASIC gene eliminated H⁺-gated currents in hippocampal neurons. In addition, ASIC null mice had impaired hippocampal long term potentiation that was rescued by enhancing NMDA receptor activity with reduced extracellular Mg²⁺ concentration or protein kinase C activation. ASIC null mice also showed deficits in learning tasks dependent upon brain regions where ASIC is normally expressed. In addition, this invention indicates that pharmacological agents that activate ASIC will likely enhance memory. Moreover, drugs that block ASIC can block the damaging affects of acidosis and excess glutamate release that occurs during seizures and strokes. Furthermore, the effects of disrupting ASIC are less severe than the effects of disrupting the NMDA receptor, medications that affect ASIC activity could be better tolerated treatments for memory loss, seizure, and the neurologic damage that results from stroke. These results suggest that acid-activated currents contribute to synaptic plasticity, learning and memory with less severe effects.

The ability of acid to activate three members of the DEG/ENaC channel family suggest they may be responsible for H⁺-gated currents in the central nervous system. Subunits of the DEG/ENaC protein family associate as homomultimers and heteromultimers to form voltage-insensitive channels. Individual subunits share a common structure with two transmembrane domains, intracellular carboxyl- and amino-termini, and a large, cysteine-rich extracellular domain thought to serve as a receptor for extracellular stimuli. Most DEG/ENaC channels are inhibited by the diuretic amiloride. The three mammalian acid-activated DEG/ENaC channels are (1) brain Na⁺ channel 1 (BNC1 (Price et al., 1996), also called MDEG (Waldmann et al., 1996), BNaCl (García-Añioveros et al., 1997), and ASIC2 (Waldmann and Lazdunski, 1998)), (2) acid sensing ion channel (ASIC (Waldmann et al., 1997b) also called BaNaC2 (García-Añoveros et al., 1997) and ASIC1 (Waldmann and Lazdunski, 1998)), and (3) dorsal root acid sensing ion channel (DRASIC (Waldmann et al., 1997a) also called ASIC3 (Waldmann and Lazdunski, 1998)). BNC1 and ASIC each have alternatively spliced isoforms (BNC1a and 1b, and ASICα and ASICβ) (Chen et al., 1998; Lingueglia et al., 1997; Price et al., 2000). Heterologous expression of most of these subunits generates Na⁺ currents that activate at low extracellular pH and then desensitize in the continued presence of acid (Waldmann and Lazdunski, 1998). Expression of individual subunits and coexpression of more than one subunit generates currents that show distinct kinetics and pH sensitivity.

Based on the transient nature of H⁺-evoked currents in primary cultures of cortical neurons and their inhibition by amiloride, Varming (Varming, 1999) suggested that DEG/ENaC channels and ASIC in particular might be responsible for the endogenous H⁺-gated currents. The pattern of expression was consistent with this idea; ASICα, BNC1a, and BNC1b have transcripts in the central nervous system (García -Añoveros et al., 1997; Waldmann et al., 1997b), whereas DRASIC and ASICβ are expressed primarily in the peripheral nervous system (Chen et al., 1998; Waldmann et al., 1997a). ASIC transcripts were most abundant in the cerebral cortex, hippocampus, cerebellum, and olfactory bulb (García-Añoveros et al., 1997; Waldmann et al., 1997b). A recent study reported that ASIC was inhibited by a peptide toxin from the venom of the South American tarantula Psalmopoeus cambridgei (Escoubas et al., 2000). This peptide also inhibited acid-evoked currents in cultured cerebellar granule cells, further suggesting that ASIC could be a component of these pH-gated currents.

There has been speculation about the physiologic and pathophysiologic function of acid-gated currents in central neurons. It has been hypothesized that interstitial acidosis associated with seizures and ischemia could trigger their activity, thereby exacerbating the pathological consequences of these conditions (Biagini et al., 2001; Ueno et al., 1992; Varming, 1999; Waldmann et al., 1997b). Although macroscopic changes in extracellular pH in the brain are tightly controlled by homeostatic mechanisms (Chesler and Kaila, 1992; Kaila and Ransom, 1998) it is possible that pH fluctuations in specific micro-domains such as the synapse may be significant (Waldmann et al., 1997b). For example, the acid pH of synaptic vesicles has been suggested to transiently influence local extracellular pH upon vesicle release (Krishtal et al., 1987; Waldmann et al., 1997b). Consistent with this idea, transient acidification of extracellular pH has been recorded with synaptic transmission in cultured hippocampal neurons (Miesenbock et al., 1998; Ozkan and Ueda, 1998; Sankaranarayanan et al., 2000) and in hippocampal slices (Krishtal et al., 1987). Thus it has been suggested that acid-evoked currents may play a role in the physiology of synaptic transmission (Krishtal et al., 1987; Waldmann et al., 1997b).

DEG/ENaC channels activated by a reduction in extracellular pH play diverse physiologic roles. The ability of these channels to respond to different stimuli and to serve different cellular functions may depend on their multimeric subunit composition, their location, associated proteins, and the cellular context. However, in the central nervous system, the function of acid-gated currents in general and H⁺-gated DEG/ENaC channels in particular has remained unknown. The present studies provide insight into the function of these channels in the central nervous system.

The discovery that ASIC contributes to acid activated currents in hippocampal neurons led to the claimed invention establishing that ASIC protein was present in the mouse brain. This result is consistent with previous reports that ASIC transcripts are present in the central nervous system (García-Añoveros et al., 1997; Waldmann et al., 1997b). Moreover, the inventors found that ASIC protein was present in the hippocampus and that acid-activated currents were missing in hippocampal neurons of ASIC −/− mice; these results indicated that ASIC is a key component of the channels that produce H⁺-gated currents. These data provide, at least in part, a molecular identity to the H⁺-gated currents that for many years have been observed in central neurons (Escoubas et al., 2000; Grantyn and Lux, 1988; Ueno et al., 1992; Varming, 1999; Vyklicky et al., 1990).

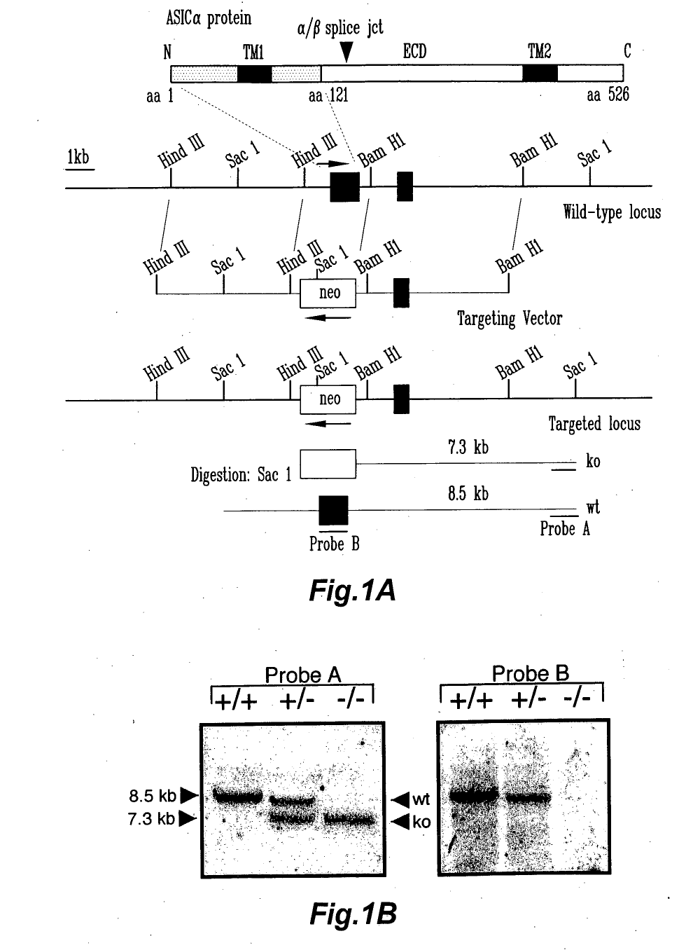

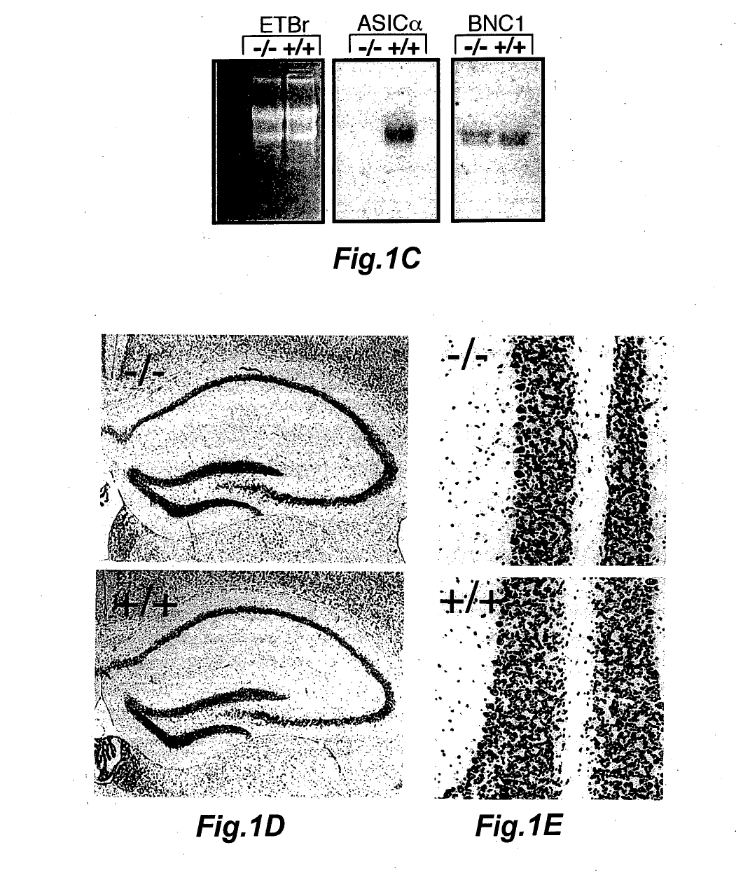

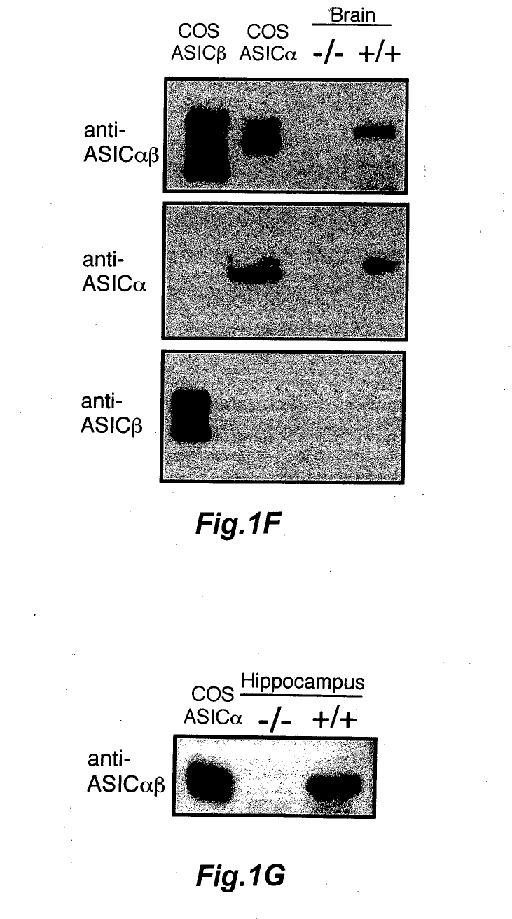

These observations also raise the question of whether ASIC is the sole subunit responsible for the H⁺-gated currents or whether other DEG/ENaC subunits might also contribute to the current. BNC1a is also expressed in hippocampal neurons (García-Añoveros et al., 1997) and unpublished observations) and BNClaRNA was expressed at normal levels in brain of ASIC −/− mice (FIG. 1C). Moreover as with ASIC homomultimers, expression of BNC1a homomultimers generates H⁺-gated currents in heterologous cells (Adams et al., 1998a; Adams et al., 1998b; Askwith et al., 2000; Bassilana et al., 1997). Therefore, it was a surprise that hippocampal neurons from ASIC null animals had no detectable transient acid-evoked current. There are at least two potential explanations. First, ASIC is the only DEG/ENaC subunit responsible for the H⁺-gated currents. Second, ASIC combines with BNC1a or other DEG/ENaC subunits to generate current, but their function depends on the presence of ASIC for some step in biosynthesis or function. Future studies will be required to explore these important alternatives.

The current data show that ASIC contributes to synaptic plasticity. The inventors found ASIC enriched in synaptosomes, immunostaining detected ASIC at synapses in a pattern suggesting primarily a dendritic localization, and paired pulse facilitation was normal in ASIC −/− hippocampal slices. These results implicated a post-synaptic localization for ASIC and suggested ASIC might play an important role in synaptic function. Although disruption of the ASIC gene did not affect basal synaptic transmission, it impaired hippocampal LTP and facilitation during HFS. Thus, ASIC was required for normal synaptic plasticity.

Several observations suggest that ASIC can contribute to LTP induction by facilitating activation of the NMDA receptor. For example, the absence of ASIC and blockade of NMDA receptors generated similar effects on EPSP facilitation during HFS. In addition, these two interventions had little effect on short-term potentiation, but impaired LTP induction (Malenka, 1991; Malenka et al., 1992). Moreover, enhancing NMDA receptor function with a low Mg²⁺ concentration or PKC activation rescued LTP in the ASIC null mice. How might ASIC influence synaptic plasticity? By generating post-synaptic Na⁺ channels it might promote membrane depolarization and the release of voltage-dependent Mg²⁺ block of the NMDA receptor, thereby facilitating a rise in intracellular Ca²⁺ concentration. Alternatively, because ASIC is slightly permeable to Ca²⁺ (Waldmann et al., 1997b), it might contribute directly to elevations of intracellular Ca²⁺.

A role in synaptic plasticity also raises a question of what ligand activates ASIC. The ability of acid to activate these channels implicates protons as the ligand (Waldmann et al., 1997b). The vesicles containing neurotransmitter are acidic (pH approximately 5.6) 2 5 (Miesenbock et al., 1998) (Sankaranarayanan et al., 2000); thus it is possible that a transient drop in synaptic pH could occur, especially with the rapid-fire release of vesicles during HFS. Transient pH reductions have been detected in extracellular fluid following repetitive nerve stimulation (Chesler and Kaila, 1992) and have been recorded in hippocampal slices during neurotransmitter release (Krishtal et al., 1987). Interestingly the rapid acid transients measured by pH sensitive dye occurred simultaneously with the EPSP waveform (Krishtal et al., 1987). Moreover, the degree of acidification was greater when elicited by a pair of sequential stimuli. This result suggests that acidification might be particularly pronounced during HFS. Although the measured acid transients were relatively small (<0.2 pH units) (Chesler and Kaila, 1992; Krishtal et al., 1987), local changes in the microenvironment of the synaptic cleft could be more pronounced.

Although, protons are the only known activators of ASIC, it is possible that other ligands may activate or modulate currents from these channels. For example, the neurotransmitter FMRFamide (Phe-Met-Arg-Phe-NH₂) activates the closely related FaNaCh channel (Lingueglia et al., 1995) which plays a role in invertebrate synaptic transmission (Castellucci and Schacher, 1990; Cottrell et al., 1992). Interestingly, FMRFamide and neuropeptide FF (NPFF) also modulate the response of ASIC channels to acid, generating a sustained component of current that follows the initial transient current (Askwith et al., 2000). Although FMRFamide has not been discovered in mammals, the mammalian brain does produce FMRFamide-related peptides, including NPFF. In rodents, central administration of FMRFamide, FMRFamide-related peptides, or antisera to these peptides alters behaviors such as learning and memory (Kavaliers and Colwell, 1993; Telegdy and Bollók, 1987). The inventors found that the effects of these peptides on learning could be mediated in part through ASIC activation. Recent data suggest that Zn²⁺ may also increase acid-evoked currents in channels composed of ASIC and BNC1α (Baron et al., 2001). The presence of high Zn²⁺ concentrations in presynaptic vesicles of hippocampal glutamatergic neurons (Slomianka, 1992) suggests that Zn²⁺ might enhance the synaptic function of these channels.

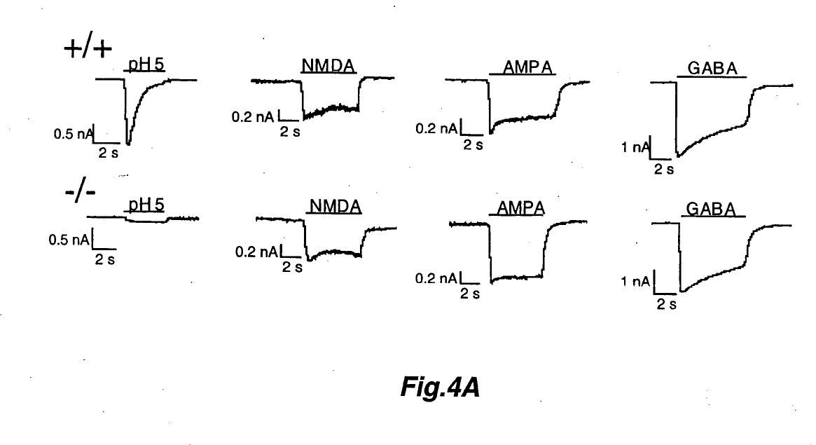

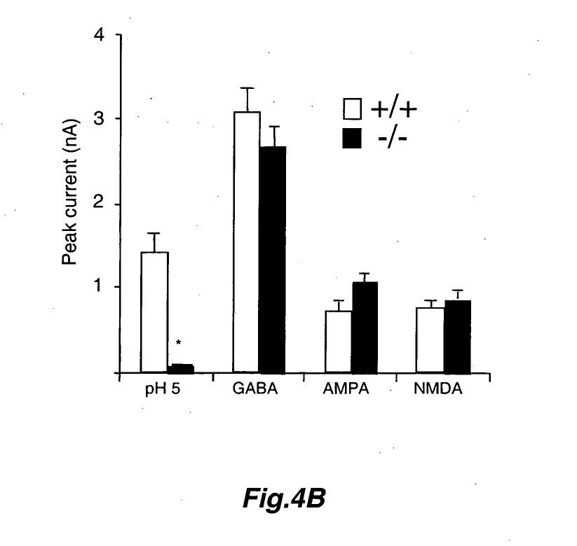

The current data also demonstrates the contribution of H⁺-gated currents to learning and memory. Our findings in the hippocampus led us to test the hypothesis that H⁺-gated channels influence learning and memory. The inventors discovered that ASIC null mice exhibited a mild deficit in spatial memory and a severe deficit in classical eyeblink conditioning. These two tasks depend on the hippocampus and cerebellum where ASIC is normally expressed ((García-Añoveros et al., 1997; Waldmann et al., 1997b) and FIG. 1) and where H⁺-gated currents have been identified ((Escoubas et al., 2000; Vyklicky et al., 1990) and FIG. 4). The relationship between hippocampal LTP and behavioral tests of learning and memory remain uncertain (for reviews see (Maren and Baudry, 1995) (Martin et al., 2000)). However in the −/− animals, the hippocampus-dependent behavioral deficit paralleled the deficit in hippocampal LTP. Increasing the stimulus intensity overcame the impairment in both cases; increasing the intensity of training overcame the behavioral defect, and reducing the Mg²⁺ concentration overcame the defect in LTP.

The degree of impairment in cerebellum-dependent eyeblink conditioning was particularly pronounced in ASIC −/− animals and comparable to that observed in Purkinje cell degeneration (pcd) mutant mice (Chen et al., 1996). Those mice exhibit a selective loss of Purkinje cells, the sole output from the cerebellar cortex, and they are functionally equivalent to animals with complete cerebellar cortical lesions. Interestingly, the pcd mice are also ataxic (Chen et al., 1996), as is often the case with impaired cerebellar function (Kim and Thompson, 1997). In contrast, ASIC null mice ambulated normally and demonstrated normal motor learning on the accelerating rotarod. Therefore, the ASIC mutation may affect only specific types of learning.

The most plausible mechanism of learning-related plasticity in the cerebellar cortex is long-term depression (LTD) between granule and Purkinje cells (Hansel et al., 2001; Maren and Baudry, 1995; Mauk et al., 1998). These cells represent a key point of convergence between the neural pathways that carry the conditioned and unconditioned stimuli. Interestingly, mature Purkinje cells do not express functional NMDA receptors (Farrant and Cull-Candy, 1991) (Llano et al., 1991). However, LTD does require post-synaptic membrane depolarization and increased post-synaptic Ca²⁺ concentrations (Daniel et al., 1998; Linden, 1994), features shared between cerebellar LTD and hippocampal LTP. As the inventors hypothesized for the hippocampus, ASIC contributes to elevations in post-synaptic Ca²⁺ concentration directly, or indirectly through membrane depolarization. A reduction in either of these processes would likely impair synaptic plasticity and memory formation in the cerebellum. Future studies will be important to elucidate the substantial impact of ASIC on cerebellum-dependent learning. In addition, ASIC −/− animals may prove to be a useful model to further explore cerebellar function.

The data indicates that ASIC would be an ideal target for pharmacological modulation of excitatory neurotransmission. Therefore, ASIC will offer a novel pharmacological target for modulating excitatory neurotransmission. For example, agents that enhance synaptic activity, such as NMDA receptor agonists have been explored as treatments to improve memory function (Muller et al., 1994). Involvement of ASIC in synaptic plasticity suggests that its activity might be manipulated for pharmacological purposes. In addition, ASIC might be inhibited to minimize the adverse consequences of acidosis. Both acidosis and high extracellular glutamate levels have been implicated in the pathology of seizures and stroke (Obrenovitch et al., 1988; Tombaugh and Sapolsky, 1990) and the NMDA receptor may play a key role in the associated excito-toxicity (Choi, 1987). NMDA receptor antagonists have been explored as treatments for these conditions, but side effects have proven intolerable (Chapman, 1998; During et al., 2000; Schehr, 1996). However, ASIC antagonists might provide a way to dampen excitatory transmission without inhibiting other key components of the system; thus ASIC antagonists might have less adverse effects than NMDA receptor antagonists. Supporting this speculation, ASIC disruption had no drastic consequences on animal development, viability, or baseline synaptic transmission. In contrast, targeted disruptions or hypomorphic alleles of the NMDA receptor are lethal or lead to severe behavioral abnormalities (Li et al., 1994; Mohn et al., 1999). Protocols for screening new drugs and drugs selected by the screening protocols will offer rich opportunities for interactions and new targets for pharmacotherapy.

The present invention provides an assay for screening compositions to identify those which are agonists, antagonists, or modulators of acid-sensing channels of the DEG/ENaC family. The assay comprises administering the composition to be screened to cells expressing acid-gated channels and then determining whether the composition inhibits, enhances, or has no effect on the channels when acid is introduced. The determination can be performed by analyzing whether a current is generated in cells containing these channels in the presence of the composition and the acid. This current can be compared to that sustained by the FMRFamide and FMRFamide-related peptides.

The foregoing and following information indicates an assay for screening compositions to identify those which are agonists, antagonists, or modulators of acid-sensing channels of the DEG/ENaC family. The assay comprises administering the composition to be screened to cells expressing acid-gated channels in the presence of acid and related peptides, and determining whether the composition enhances or inhibits the opening the acid-sensing ion channels of the DEG/ENaC channel family. In addition to the ASIC channels, it is expected that FMRFamide or FMRFamide related peptides will potentiate acid-evoked activity of other members of the DEG/ENaC cation channel family. The determination of enhancement or inhibition can be done via electrophysical analysis. Cell current can be measured. Alternatively, any indicator assay which detects opening and/or closing of the acid-sensing ion channels can be used such as voltage-sensitive dyes or ion-sensitive dyes. An assay which caused cell death in the presence of the peptide, or agonist, would be the most definitive assay for indicating potentiation of the channels. Assays which could measure binding of FMRFamide and related peptides to the channels could identify binding of agonists, antagonists, and modulators of binding. One of ordinary skill in the art would be able to determine or develop assays which would be effective in finding compositions which effect the acid-sensory ion channels. A composition which activates or inactivates the transient or sustained current present when acid or a related peptide activate the acid-sensing ion channels should be useful as a pharmacological agent. The screening can be used to determine the level of composition necessary by varying the level of composition administered. The composition can be administered before or after addition of the acid or a related peptide to determine whether the composition can be used prophylactically or as a treatment for enhanced synaptic plasticity, learning or memory. One of ordinary skill in the art would be able to determine other variations on the assay(s).

Suitable formulations for parenteral administration include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, include for example, sodium carboxymethyl cellulose, sorbitol and/or dextran, optionally the suspension may also contain stabilizers. In addition to administration with conventional carriers, active ingredients may be administered by a variety of specialized delivery drug techniques which are known to those of skill in the art. The following examples are given for illustrative purposes only and are in no way intended to limit the invention.

Compositions which bind to the channels can be identified or designed (synthesized) based on the disclosed knowledge of potentiation of the channels and determination of the three-dimensional structure of the channels. These compositions could act as agonists, antagonists, or modulators effecting synaptic plasticity, learning, memory or other physiological responses.

In general, in addition to the active compounds, i.e. the ASIC agonists and antagonists, the pharmaceutical compositions of this invention may contain suitable excipients and auxiliaries which facilitate processing of the active compounds into preparations which can be used pharmaceutically. Oral dosage forms encompass tablets, dragees, and capsules. Preparations which can be administered rectally include suppositories. Other dosage forms include suitable solutions for administration parenterally or orally, and compositions which can be administered buccally or sublingually.

The pharmaceutical preparations of the present invention are manufactured in a manner which is itself well known in the art. For example the pharmaceutical preparations may be made by means of conventional mixing, granulating, dragee-making, dissolving, lyophilizing processes. The processes to be used will depend ultimately on the physical properties of the active ingredient used.

Suitable excipients are, in particular, fillers such as sugars for example, lactose or sucrose mannitol or sorbitol, cellulose preparations and/or calcium phosphates, for example, tricalcium phosphate or calcium hydrogen phosphate, as well as binders such as starch, paste, using, for example, maize starch, wheat starch, rice starch, potato starch, gelatin, gum tragacanth, methyl cellulose, hydroxypropylmethylcellulose, sodium carboxymethylcellulose, and/or polyvinyl pyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. Dragee cores may be provided with suitable coatings which, if desired, may be resistant to gastric juices.

For this purpose concentrated sugar solutions may be used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments may be added to the tablet of dragee coatings, for example, for identification or in order to characterize different combination of compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin, or liquid polyethylene glycols. In addition stabilizers may be added. Possible pharmaceutical preparations which can be used rectally include, for example, suppositories, which consist of a combination of the active compounds with the suppository base. Suitable suppository bases are, for example, natural or synthetic triglycerides, paraffinhydrocarbons, polyethylene glycols, or higher alkanols. In addition, it is also possible to use gelatin rectal capsules which consist of a combination of the active compounds with a base. Possible base material includes for example liquid triglycerides, polyethylene glycols, or paraffin hydrocarbons.

Suitable formulations for parenteral administration include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, include for example, sodium carboxymethyl cellulose, sorbitol and/or dextran, optionally the suspension may also contain stabilizers.

In addition to administration with conventional carriers, active ingredients may be administered by a variety of specialized delivery drug techniques which are known to those of skill in the art. The following examples are given for illustrative purposes only and are in no way intended to limit the invention.

In conclusion, these results indicate that acid-activated channels influence synaptic plasticity, learning and memory. Further, elucidation of the mechanisms that control ASIC activity and the connection between H⁺-gated channels and behavior should provide new insight and treatments for synaptic function and the processes that underlie synaptic plasticity, learning and memory.

Definitions

For purposes of this application the following terms shall have the definitions recited herein. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUM Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The term “antibody” includes reference to antigen binding forms of antibodies (e.g., Fab, F(ab)₂). The term “antibody” frequently refers to a polypeptide substantially encoded by an immunoglobulin genes, or fragments thereof which specifically bind and recognize an analyte (antigen). However, while various antibody fragments can be defined in terms of the digestion of an intact antibody, one of skill will appreciate that such fragments may be synthesized de novo either chemically or by utilizing recombinant DNA methodology. Thus, the term antibody, as used herein, also includes antibody fragments such as single chain Fᵥ, chimeric antibodies (i.e., comprising constant and variable regions from different species), humanized antibodies (i.e., comprising a complementarily determining region (CDR) from a non-human source) and heteroconjugate antibodies (e.g., bispecific antibodies).

As used herein the term “ASIC receptor activator” includes any compound which causes activation of the ASIC receptor. This includes both competitive and non-competitive agonists as well as prodrugs which are metabolized to ASIC agonists upon administration, as well as analogs of such compounds shows by the assays herein to be active ASIC agonists.

As used herein the term “ASIC receptor blocker” includes any compound which causes inhibition of the ASIC receptor. This includes both competitive and non-competitive antagonists as well as prodrugs which are metabolized to ASIC antagonists upon administration, as well as analogs of such compounds shows by the assays herein to be active ASIC antagonists.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring acids is that, when incorporated

into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of posttranslation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention.

As used herein the term “therapeutically effective” shall mean an amount of ASIC receptor blocker or activator, depending upon the condition being treated, to block the effect of the ASIC receptor as determined by the methods and protocols disclosed herein.

### EXAMPLES

To understand the role of acid-gated currents in central neurons in general, and the role of ASIC in particular, the inventors generated mice with a targeted disruption of the ASIC gene. The inventors then examined how ASIC contributes to neuronal acid-gated currents and to synaptic function and behavior.

Methods and Materials

Generation of ASIC Knockout Mice

The results were determined by the generation of ASIC knockout mice as described in the following model. This animal model can be used for predicting success in humans. ASIC knockout mice were generated by homologous recombination in embryonic stem cells using an approach similar to that previously reported (Price et al., 2000). A 17 kb genomic clone containing a portion of the ASIC gene was obtained by screening a lambda bacteriophage library of mouse strain SV129 genomic DNA. The wild-type locus, targeting vector and targeted locus are shown schematically in FIG. 1A. In the knockout allele, a PGK-neo cassette replaces the first exon of the ASIC gene and approximately 400 bp of upstream sequence. The deleted exon encodes amino acids 1-121 of mASICα. The neo cassette introduced a new Sac I restriction enzyme site, which was used to screen for targeted integration of the vector. The wild-type and knockout alleles were identified in stem cell clones and in mice by Southern blotting Sac I digested genomic DNA with oligo-labeled cDNA probes corresponding to a 1 kb region that flanks the sequence contained in the targeting vector or with a cDNA probe corresponding to the disrupted sequence. Genotyping was performed by isolating genomic DNA from tail snippets by PCR using the following primers: wild type allele (5′-CCGCCTTGAGCGGCAGGTTTAAAGG-3′; 5′-CATGTCACCAAGCTCGACGAGGTG-3′), knockout allele (5′-CCGCCTTGAGCGGCAGGTTTAAAGG-3′; 5′TGGATGTGGAATGTGTGCGA-3′). Northern blotting was performed using the disrupted exon of ASIC as a cDNA oligo-labeled probe against equivalent amounts of total brain RNA. BNC1 RNA expression levels were determined using a probe described previously (Price et al., 2000). Brain histology was performed on mouse brains removed following halothane anesthesia and whole body perfusion with 4% formaldehyde. Brains were fixed overnight, embedded in paraffin, cut into 6 μm sections and stained for Nissl substance with crystal violet acetate.

Antibodies The anti-ASICαβ antibody was generated by injecting rabbits with a bacterially expressed thioredoxin fusion protein from pET32b (Novagen) containing the amino acid sequence EVIKHKLCRRGKCQKEAKRSSADKGVALSLDDVKRHNPCESLRGHPAGMTYAANI LPHHPARGTFEDFTC corresponding to the extreme carboxyl-terminus of hASIC (Pocono Rabbit Farm & Laboratory, Inc.). The anti-ASICα, and anti-ASICβ antibodies were generated by injecting sheep with the synthesized peptides MELKTEEEEVGGVQPVSIQAFA or MELDEGDSPRDLVAFANSCTLH which correspond to the first 22 amino acids of mAISCα and mASICβ respectively (Elmira Biologicals). Affinity purified antibodies were generated by absorbing sera to the specific immunogen coupled to Affi-Gel 10 or Affi-Gel 15 (Bio-Rad), washing with PBS, eluting with 50 mM glycine-HCl pH2.5, neutralizing with Tris buffer pH 10.4, and stored in 1% BSA/PBS at 4° C. or −20° C. Anti-PSD-95 monoclonal and anti-GluR2/3 antibodies were used according to the recommendations of the manufacturer (Sigma).

Immunoprecipitation, Immunoblotting, Subcellular Fractionation Immunoblotting and immunoprecipitation. Cos-7 cells transfected by electroporation (mASICα or mASICβ subcloned as Cla I-Kpn I fragments into pMT3), whole mouse brains, or dissected hippocampi were homogenized in homogenization buffer (HB: phosphate buffered saline (PBS) with 1% Triton X-100 and protease inhibitors-1 mM EDTA, 0.4 mM phenylmethylsulfonyl fluoride, 20 μg/ml aprotinin, 20 μg/ml leupeptin, 10 μg/ml pepstatin A). Following homogenization the protein extracts were subjected to a 700 x g spin to remove large organelles and particulate debris. This represents the “total protein extract”. This extract was subjected to SDS-PAGE for western blotting with the indicated antibodies or used for immunoprecipitation. For immunoprecipitation, 1 μl of undiluted affinity purified αASIC-6.4 antibody was added to 750 μl of total protein extract in HB and incubated overnight with agitation at 4° C. Protein A sepharose 50 μl (Pierce, 15 mg/ml) was added and further incubated for 1 hr at 4° C. Immunoprecipitates were precipitated at 14 k rpm in a microfuge (Eppendorff) and washed three times with HB, resuspended in sample buffer (0.125 mM Tris, pH 7.5, 3.4% SDS, 17% glycerol, 67 mM dithiothreitol, 0.008% bromphenol blue), boiled 5 min. and western blotted with the indicated antibodies. For western blots or immunoprecipitation, equivalent amounts of protein extract were determined based on the amount of starting material or by Lowry protein assay (Lowry and Passanneau, 1972). Synaptosomal fractionation. The synaptosomal fraction was prepared as described Torres et al. with modification (Torres et al., 1998). One adult mouse brain was homogenized in 3.6 ml synaptosome homogenization buffer (SHB: 320 mM sucrose, 4 mM HEPES (pH 7.4), 1 mM EGTA, 0.4 mM phenylmethylsulfonyl fluoride, 20 μg/ml aprotinin, 20 μg/ml leupeptin, 10 μg/ml pepstatin A) with 10 up/down strokes of a tight fitting glass dounce tissue grinder (Wheaton 7 ml). The crude homogenate was centrifuged at 1,000×g for 10 min. The supernatant was collected and centrifuged at 12,000×g for 15 min., and the second pellet was resuspended in 2.5 ml SHB and centrifuged at 13,000×g for 15 min. The resulting pellet representing the synaptosomal fraction (SF) was resuspended in 0.5 ml of SHB. Protein concentration determined with the Biorad Protein Assay. Equivalent amounts of protein (10 μg for PSD95 and GluR2/3; and 200 μg for ASIC) from the crude homogenate and the SF were separated on SDS-PAGE gels, and western blot analyses were performed with antibodies to the indicated proteins.

We were unable to detect the endogenous ASIC protein in the brain and CNS neurons by immunocytochemistry (not shown). This has been noted by others who have suggested that this problem may be due to low levels of protein expression and/or epitope masking (Olson et al., 1998).

Hippocampal Neuron Cultures, Plasmid Transfection, and Immunofluorescent Staining

Mouse hippocampal cultures were generated from postnatal day 1-2 pups according to the method of Mennerick et. al. (Mennerick et al., 1995). Hippocampi were dissected, separated into pieces, and enzymatically dissociated in 1 mg/ml papain in oxygenated Leibovitz's L-15 medium, 20 min., 37° C. Cells were triturated and plated on slides or coverslips coated with 0.5 mg/ml rat tail collagen. Culture media consisted of Earle's MEM supplemented with 5% horse serum, 5% fetal calf serum, 17 mM glucose, 400 μM glutamine, 50 U/ml penicillin, 50 mg/ml streptomycin, and insulin-transferrin-sodium selenite media supplement (Sigma I-1884, resuspended in 50 mls H₂O, 2.5 μl was added per ml of media). After 3-4 days in culture, cells were treated with 10 μM cytosine arabinoside to halt glial proliferation.

Glia-free rat hippocampal neurons from embryonic day 18 pups were purchased from Brain Bits, Springfield, IL (Brewer, 1997). Neurons were stored at 4° C. for up to 1 week prior to plating. They were triturated and resuspended in media (B27/Neurobasal supplemented with 0.5 mM glutamine, 25 μM glutamate) and plated on poly-L-lysine coated glass coverslips in 24-well plates. One-half volume of media (minus glutamate) was changed every 4-5 days.

Rat neurons in primary culture for 4-8 days were transfected using the calcium phosphate method of Xia et al (Xia et al., 1999), with 1.6 μg plasmid DNA expressing ASIC in combination with an equal amount of pGreen Lantern-1 (Gibco BRL) or PSD-95-GFP (kind gift of D. Bredt (Craven et al., 1999). For expression in neurons hASIC was subcloned as a Not I, Kpn I fragment into pcDNA3.1 (Invitrogen). ASIC-FLAG was generated by PCR mutagenesis inserting the Flag epitope DYKDDDK at the extreme N-terminus of hASIC and subcloned into pcDNA3.1

Hippocampal neurons in culture for 8-14 days were used for immunocytochemistry. Cells were fixed at room temperature for 10-15 min. (PBS plus 4% formaldehyde, 4% sucrose), permeabilized (0.25% Triton X-100 in PBS) 5 min. at room temp, washed twice for 5 min. in PBS, and incubated at room temp. for 2 hr with the M2 monoclonal anti-Flag antibody (International Biotechnologies, 1:600) diluted in 3% BSA/PBS. Cells were washed again in PBS 3 times for 5 min., and incubated for 1 hr at 37° C. with Cy3-conjugated anti-mouse antibody (Jackson ImmunoResearch, Inc., 1:300). Cells were washed again in PBS, mounted with Vectashield (Vector Labs) and visualized with a Bio-Rad 1024 scanning confocal microscope (Bio-Rad, Hercules, Calif.).

Hippocampal Slice Recordings

Transverse hippocampal slices (350-400 μm) were prepared from wild type (+/+) and ASIC knock out (—/—) littermates at 2-4 months of age. For the LTP studies, the applicant was blinded to genotype. The slices were sectioned in ice-cold artificial cerebrospinal fluid (ACSF) containing (in mM) 119 NaCl, 2.5 KCl, 2.5 CaCl2, 1.0 NaH₂PO4, 1.3 MgSO4, 26.2 NaHCO₃, 11 glucose, pH 7.4, bubbled with 95% O₂/5% CO₂, and then were incubated in identical solution at 31° C. for 2-5 hours before recording.

Standard extracellular field potential recording techniques were used. Experiments were performed in a submerged chamber, heated to 31±0.5° C. Field postsynaptic excitatory potentials (EPSPs) were evoked in CA1 stratum radiatum by stimulation of Schaffer collaterals with a bipolar stainless steel electrodes that was put at the border of CA3-CA1 subfields and recorded with 3M NaCl-filled glass pipettes (<5 MΩ) using a biological amplifier (WPI, Iso-DAM8, FL., USA). A 100 μs test stimulation was delivered every 30 s by a stimulus isolation unit (Grass, SD9, Mass., USA). Input-output curves were obtained by plotting the stimulus voltage against the amplitude of EPSPs. Only slices exhibiting EPSPs of≧1 mV in amplitude were examined further. Stimulus intensity was adjusted to evoke half-maximal responses. LTP was induced by a high-frequency stimulation (HFS, 100 Hz, 1 s, at test intensity). Paired-pulse facilitation (PPF) was observed by applying paired pulses with different intervals (20, 50 ms). LTP was measured by normalizing the EPSP slopes after HFS to the mean slope of the baseline EPSP before HFS. Data were digitized (10 kHz), filtered at 1 kHz (eight-pole Bessel Filter), monitored on-line, and stored on hard disk using PULSE 8.41 (HEKA, Lambrecht, Germany). Off-line analysis was performed by PATCHMACHINE 1.0 (http://www.hoshi.org) and IGORPRO 4.0 (WaveMetrics, Lake Oswego, Oreg., USA). Unless otherwise noted, two-sample t-test was used to calculate statistical significance.

### Example 1

### Targeted Disruption of the Mouse ASIC Gene

ASIC knockout mice were generated by deleting a region of genomic DNA encoding the first 121 amino acids of ASICα. This region includes the intracellular N-terminus, the first transmembrane domain, and a portion of the extracellular domain of the ASICα protein. The wild-type locus, targeting vector and targeted locus are shown schematically in FIG. 1A. Southern hybridization of Sac 1 digested genomic DNA with the flanking probe demonstrated targeted integration (FIG. 1B, probe A). Southern hybridization using the targeted exon as a probe confirmed the elimination of this sequence in the knockout mice (FIG. 1B, probe B). Consistent with the absence of a critical portion of the ASIC gene, there was a disruption of the corresponding message in total brain RNA by northern blotting (FIG. 1C). In contrast, the level of BNC1 transcripts was unchanged in ASIC −/− brain relative to +/+ littermates (FIG. 1C).

ASIC knockout mice were viable and indistinguishable in size and appearance from wild-type littermates. The −/− mice were fertile, had a normal life span, and had no apparent abnormalities in movement or ambulation. There were no noticeable anatomic abnormalities in the −/− mice. Moreover, there were no apparent differences in brain morphology and no differences in neuron appearance and distribution in the hippocampus (FIG. 1D) or cerebellum (FIG. 1E) of the −/− mice relative to controls.

The inventors tested for ASIC protein in brain using an antibody against the intracellular carboxyl-terminus (anti-ASICαβ); this antibody recognizes both ASICα and ASICβ expressed in transfected COS cells (FIG. 1F). Immunoprecipitation and western blotting detected ASIC in protein extracts of whole brain and hippocampus of +/+ but not −/− animals (FIG. 1F, G). Protein was also detected when anti-ASICαβ immunoprecipitates were probed with an antibody specific for ASICα (anti-ASICα), but not with an antibody specific for ASICβ (anti-ASICβ). These data suggest that the ASICα isoform is much more abundant in mouse brain than ASICβ. This observation agrees with the previous finding that ASICβ transcripts are not detected in the rat brain (Chen et al., 1998). These data also show the loss of ASIC protein in −/− animals.

### Example 2

### ASIC Colocalizes with PSD-95 in Hippocampal Neurons and Synaptosome-Enriched Subcellular Fractions

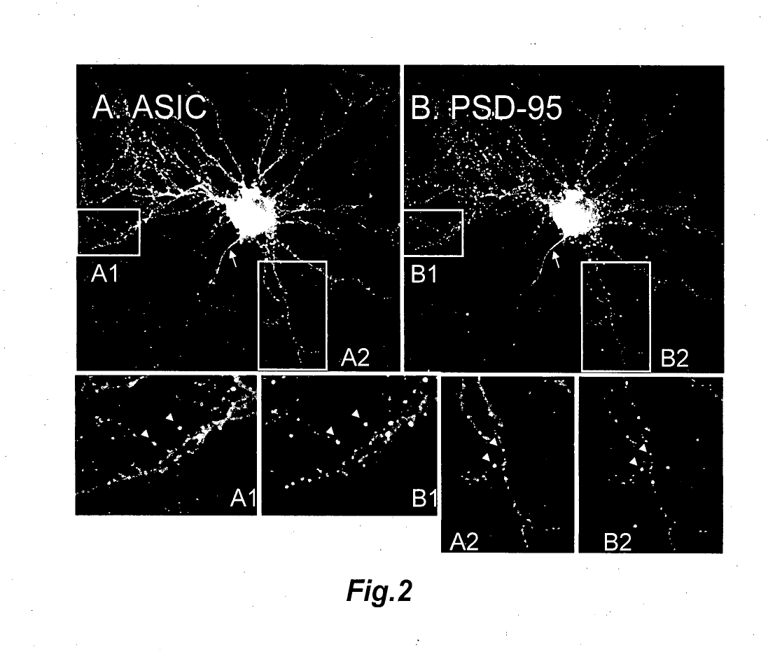

To investigate the location of ASIC within neurons, cultured hippocampal neurons were transfected with an epitope-tagged ASICα and studied its distribution by immunocytochemistry. ASIC specific immunostaining was detected in the cell body and in a punctate pattern in dendritic processes both proximally and distally (FIG. 2A). The distribution of ASIC in axons (FIG. 2A, arrow) was less pronounced and more diffuse. The localization of ASIC coincided in large part with that of co-transfected PSD95 linked to GFP (FIG. 2B); this fusion protein exhibits a synaptic pattern of distribution (Craven et al., 1999). GFP alone distributed diffusely throughout the neuron and the pattern of ASIC distribution was not dependent upon exogenous PSD-95 expression (not shown). These results suggest that ASIC is located at hippocampal synapses, particularly in the postsynaptic membrane.

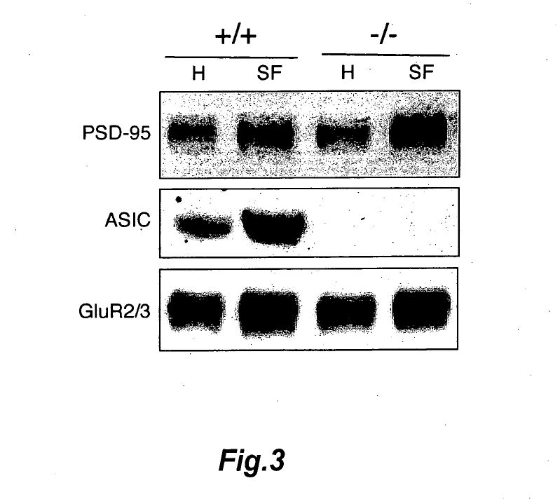

To explore whether endogenously expressed ASIC protein is distributed similarly to PSD-95 in the brain, inventors prepared synaptosome-enriched subcellular fractions of brain from wild type and knockout mice. As described by others (Cho et al, 1992; Xia et al., 1999), these fractions are enriched in both pre- and postsynaptic proteins. Both PSD-95 and GluR2/3 are increased in synaptosome-enriched fractions (Cho et al., 1992; Xia et al., 1999). Likewise, ASIC protein showed substantial enrichment in the synaptosome-containing fractions (FIG. 3). These data support the results obtained by immunostaining (FIG. 2) and suggest that ASIC is present at synapses.

### Example 3

### ASIC Contributes to Acid-Evoked Currents in Hippocampal Neurons

Previous studies have identified acid-evoked Na⁺ currents in hippocampal neurons (Vyklicky et al., 1990). The presence of ASIC in these neurons suggested that this channel subunit contributes to the H⁺-gated currents. To test this hypothesis, the currents in cultured hippocampal neurons by whole-cell patch-clamp were measured.

Whole-cell patch-clamp was performed on large hippocampal pyramidal neurons cultured for 1 to 2 weeks. Electrodes had a resistance of 4-7 MΩ when filled with the intracellular solution containing (in mM): 120 KCl, 10 NaCl, 2 MgCl₂, 5 EGTA, 10 4-(2-hydroxyethyl)-1-piperazineethanesulfonic acid (HEPES) and 2 ATP. The pH was adjusted to 7.2 with tetramethylammonium hydroxide (TMA-OH) and osmolarity with tetramethylammonium chloride (TMA-Cl). Extracellular solutions contained (in mM): 128 NaCl, 1.8 CaCl₂, 5.4 KCl, 5.55 glucose, 10 HEPES and 10 2-(4-morpholino)-ethanesulfonic acid (MES), and 1 μM tetrodotoxin. pH was adjusted to 7.4 or 5 with TMA-OH and osmolarity normalized with TMA-Cl. Neurons were held at −80 mV during recording. Solutions were changed by directing the flow from the appropriate perfusion pipe to the neuron. Data was acquired at 2 kHz with an Axopatch 200B amplifier using a 0.5 kHz low pass filter and Clampex 8.0 softwear (Axon, Foster City, Calif.).

Consistent with previous findings (Vanming, 1999; Vyklicky et al., 1990), the inventors found that application of acid generated a transient current in neurons from wild-type mice (FIG. 4A). Of wild-type neurons, 93% (n=76) exhibited these currents. In striking contrast, acid failed to activate analogous currents in neurons from −/− mice (n=99). Although loss of ASIC abolished H⁺-gated currents, the currents activated by GABA, AMPA, and NMDA appeared normal in −/− neurons (FIG. 4A, B). These data indicate that ASIC is a required component of the channels that respond to acid in hippocampal neurons.

### Example 4

### Baseline Synaptic Transmission in the Hippocampus is Normal in ASIC Knockout Mice

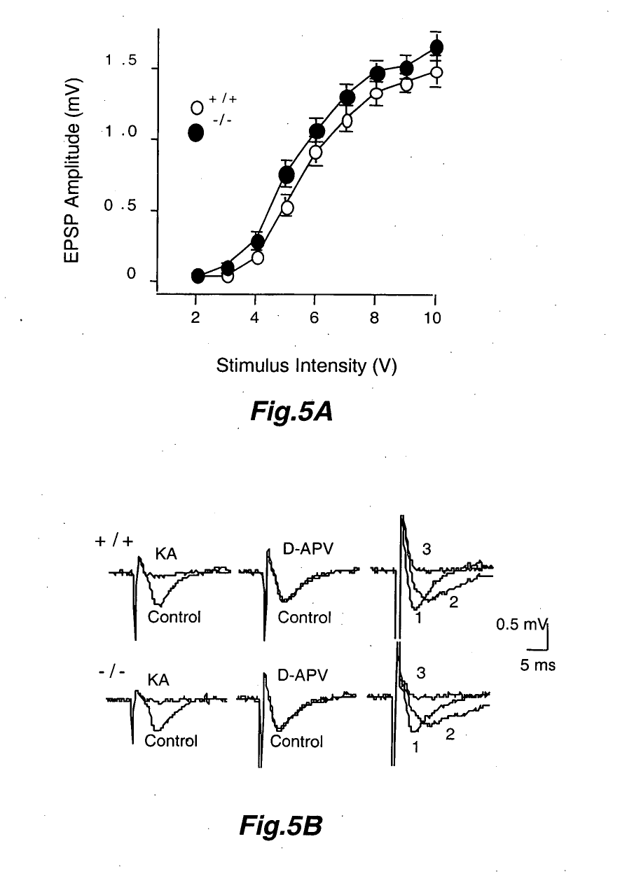

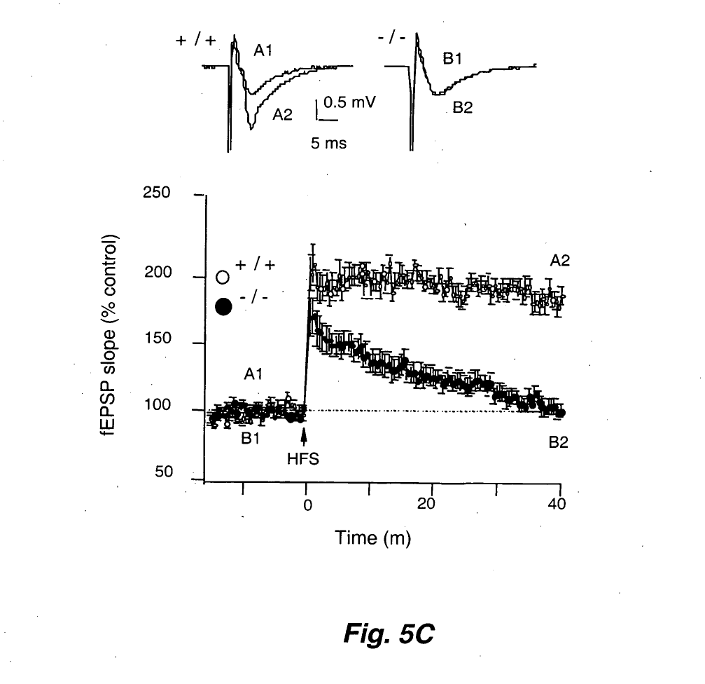

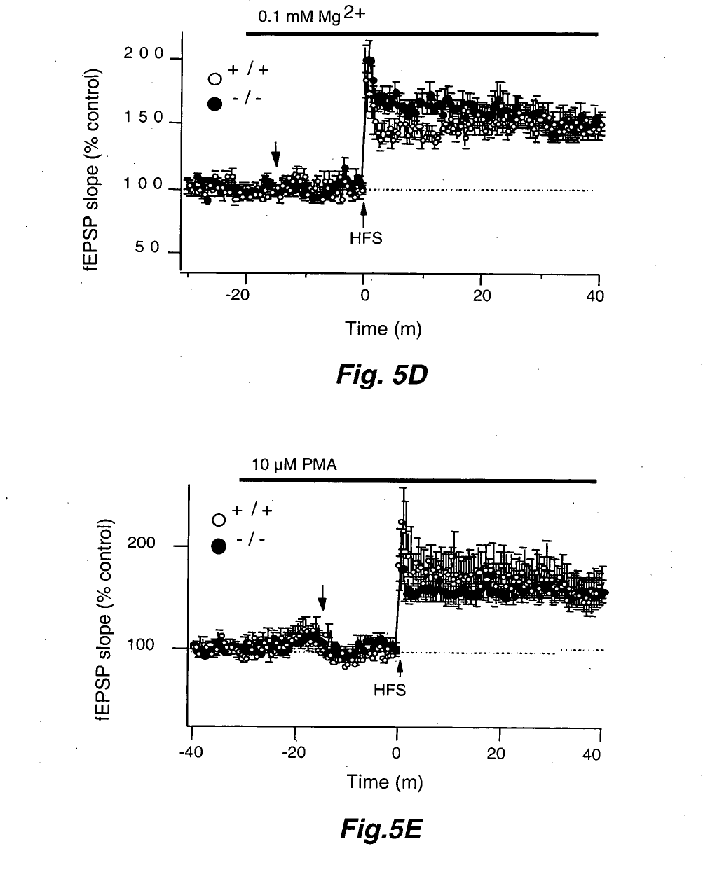

The absence of H⁺-gated currents in hippocampal neurons of −/− animals provided the opportunity to assess their physiologic significance in the hippocampus. To explore the potential function of these channels at hippocampal synapses, synaptic transmission at Schaffer collateral-CA1 synapses in hippocampal slices was tested. Field excitatory post-synaptic potentials (fEPSPs) at baseline were similar in slope and amplitude between −/− and +/+ mice (FIG. 5A, B). In addition, the fEPSP amplitude did not differ significantly between −/− and +/+ mice with increases in stimulus intensity (FIG. 5A). Likewise, the components of the EPSP mediated by AMPA and NMDA did not differ between genotypes when dissected out by selective antagonists (FIG. 5B). These results suggested that synaptic transmission at baseline was not affected by the loss of ASIC.

### Example 5

### Impairment of Long-Term Potentiation in ASIC Knockout Mice

To examine whether other aspects of synaptic function could be affected by disrupting ASIC, the inventors tested long-term potentiation (LTP) in the hippocampus (Bliss and Collingridge, 1993; Malenka and Nicoll, 1999) (Malinow et al., 2000). LTP at Schaffer collateral-CA1 synapses represents one form of synaptic plasticity and serves as a molecular model for specific types of learning and memory (Bliss and Collingridge, 1993) (Shimizu et al., 2000). Immediately following LTP induction with high frequency stimulation (HFS) slices from both genotypes showed an increase in fEPSP slope and amplitude. This result suggests that short-term potentiation occurs in both groups (FIG. 5C), although the degree of short-term potentiation was slightly less in the −/− group. In contrast, long-term potentiation was strikingly impaired in the −/− mice. By 40 min. after HFS the fEPSPs from −/− mice had decayed to baseline whereas fEPSPs from +/+ mice remained potentiated. This result indicates that ASIC and H⁺-gated currents may play a specific role in the development or maintenance of LTP.

A central feature of CA1 LTP is activation of the NMDA receptor due to binding of the neurotransmitter glutamate and to depolarization of the postsynaptic membrane through the release of voltage-dependent Mg²⁺ block (Malenka and Nicoll, 1999) (Bliss and Collingridge, 1993; Malinow et al., 2000). To determine whether the loss of ASIC might impact this process, the LTP experiments with a low Mg2+ concentration (0.1 mM) in the bathing solution were repeated. Previous work has shown that low Mg²⁺ concentrations facilitate LTP by promoting activation of the NMDA receptor (Huang et al., 1987). Following HFS in the presence of low Mg²⁺, both genotypes exhibited comparable LTP (FIG. 5D). Thus the reduced Mg2+ concentration restored LTP in the −/− slices (FIG. 5D). This result suggests that facilitating NMDA receptor function may be sufficient to overcome the ASIC-dependent deficit in LTP.

Another component of LTP generation is activation of PKC (Malinow et al., 1989) (Ben-Ari et al., 1992; Wang and Feng, 1992). One effect of PKC activation in the CA1 area of the hippocampus is on the Ca²⁺-dependent regulation of the NMDA receptor (Chen and Huang, 1992; Hisatsune et al., 1997; Lu et al., 2000). As observed previously (Malenka et al., 1986), when the PKC in the brain slice was activated by the addition of phorbol esters potentiation in EPSP slope and amplitude was observed. However, inventors discovered that by adjusting the dose of phorbol 12-myristate 13-acetate (PMA) to 10 μM, and in a few cases turning down the stimulus intensity, a stable baseline in transmission could be achieved (FIG. 5E). Following HFS in the presence of PMA, LTP was restored in the −/− slices (FIG. 5E). Like the experiments using a low Mg²⁺ concentration, this result suggests that in the absence of ASIC, LTP induction may require the enhancement of another component of the system.

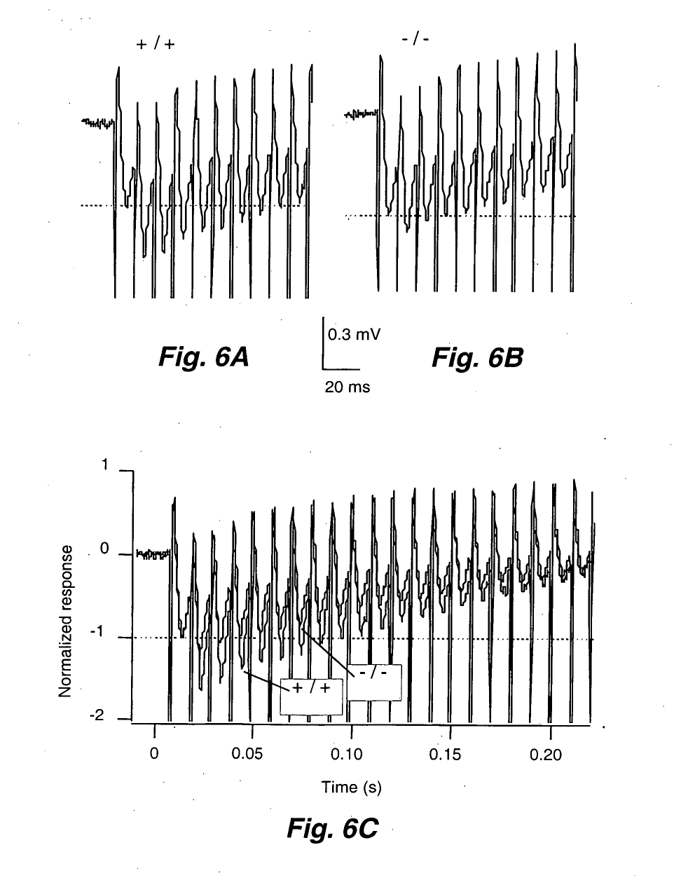

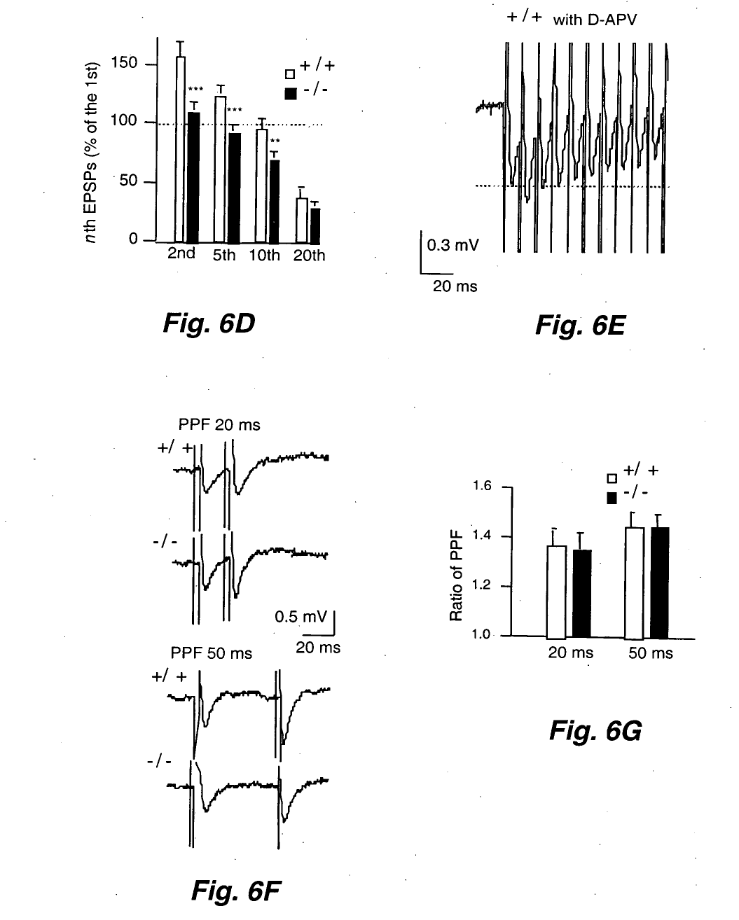

Activation of the NMDA receptor during HFS is critical for LTP induction; for example, a partial blockade of the NMDA receptor with D-APV prevents LTP but spares short-term potentiation (Malenka, 1991). This is similar to our results in which the loss of ASIC prevented LTP, but not short-term potentiation (FIG. 5C). Our data showing that LTP can be rescued in the ASIC −/− mice by amplifying NMDA receptor function (FIG. 5D, E) suggested that hypothesis that ASIC may contribute to NMDA receptor activation during LTP induction. Therefore fEPSPs during high frequency stimulation were examined. In wild type slices, fEPSP amplitude was facilitated during the initial period of HFS; relative to the first EPSP the amplitudes of the next 7 EPSPs were increased (FIG. 6A). In ASIC null mice the facilitation during HFS was markedly attenuated (FIG. 6B-D). To investigate whether inadequate NMDA receptor activation could account for the impaired facilitation, the inventors applied D-APV to wild type slices prior to HFS. The pattern of EPSP facilitation elicited by blocking the NMDA receptor showed a remarkable resemblance to that obtained in ASIC null slices (FIG. 6B, E). Thus ASIC-dependent facilitation of NMDA receptor function could account for the impact of ASIC on LTP.

### Example 6

### Paired Pulse Facilitation is Normal in ASIC Null Mice

Paired pulse facilitation serves as a commonly used index of presynaptic activity and neurotransmitter release probability (Pozzo-Miller et al., 1999; Schulz et al., 1994). The inventors found comparable paired pulse facilitation in animals of both genotypes (FIG. 6F, G). Moreover as expected, D-APV had no effect on paired pulse facilitation (not shown). These experiments suggest that presynaptic neurotransmitter release is normal in the ASIC knockout mice.

### Example 7

### ASIC Null Mice Exhibit a Mild and Reversible Deficit in Spatial Learning and Memory

NMDA receptor-dependent synaptic plasticity in the CA1 region of the hippocampus has a key role in the acquisition and consolidation of spatial memory (Tsien et al., 1996) (Shimizu et al., 2000). Impaired synaptic plasticity in ASIC knockout mice suggested they might show a defect in hippocampus-dependent spatial learning. To test this the hidden platform version of the Morris water maze was used (Morris, 1981).

The protocol was similar to that used previously by others (Abeliovich et al., 1993). A seamless galvanized metal pool 1.2 m in diameter and 0.6 m high was painted drab green and filled to a height of 0.4 m with water made opaque with non-toxic crayola paint. A platform 0.11 m in diameter and 0.39 m high was constructed by capping the ends of a lead-filled fiberglass pipe and painted the same color as the pool so that it was not visible when submerged 1 cm below the water surface. The platform was placed into the center of a quadrant so that the closest edge was 10 cm from the wall of the pool. The four quadrants of the pool were designated N, S, E, and W. Four starting locations NE, SE, SW, and NW were designated at the edge of the wall of the pool at the intersections between the quadrants. The location of the platform stayed the same for each mouse but varied between mice. Before the start of training, naïve mice were given a 60 s practice swim and 3 practice attempts at climbing onto the platform. A trial consisted of placing the mouse in the pool facing the wall at one of the 4 starting locations. It was then released and given up to 60 s to find the platform. Once the animal climbed onto the platform it was allowed to remain for 30 s. Animals that did not climb onto the platform in 60 s were manually guided to the platform and allowed to climb on. Following 30 s on the platform, the animal was either returned to the home cage or another trial initiated. Two training protocols were used. In the first protocol, mice were given a single trial per day for 11 consecutive days. The second protocol consisted of 3 blocks of 4 trials per day for 3 consecutive days. The probe trials were similar to training trials except the platform was removed from the pool. Escape latency, time spent in quadrants, and number of platform crossings were scored by an observer blinded to genotype from videotape recordings of the individual trials.

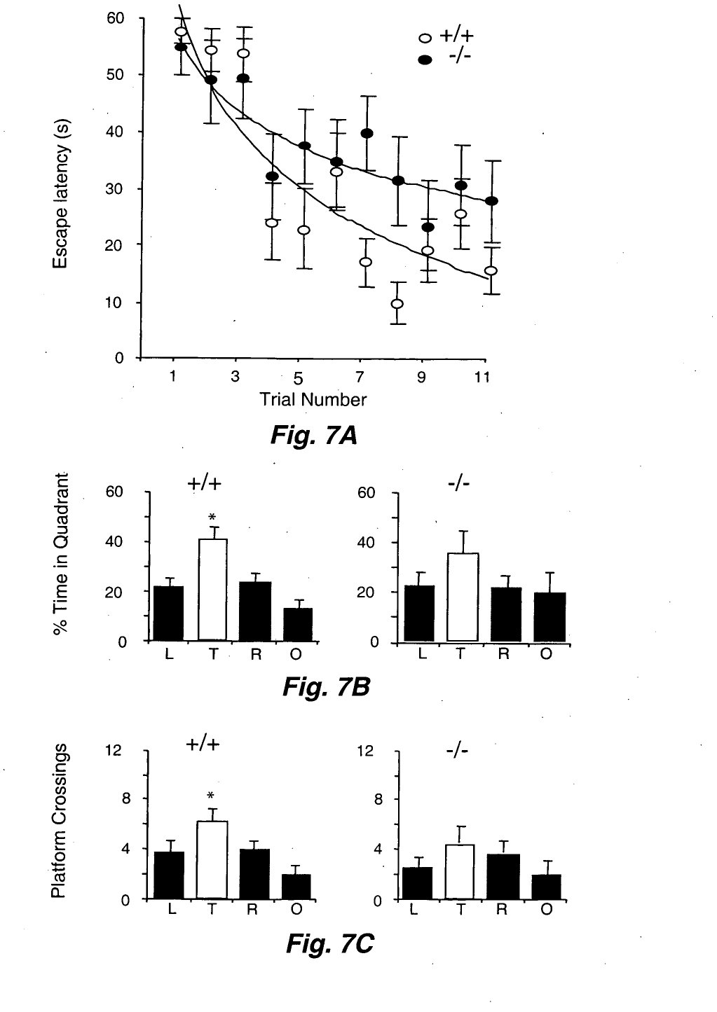

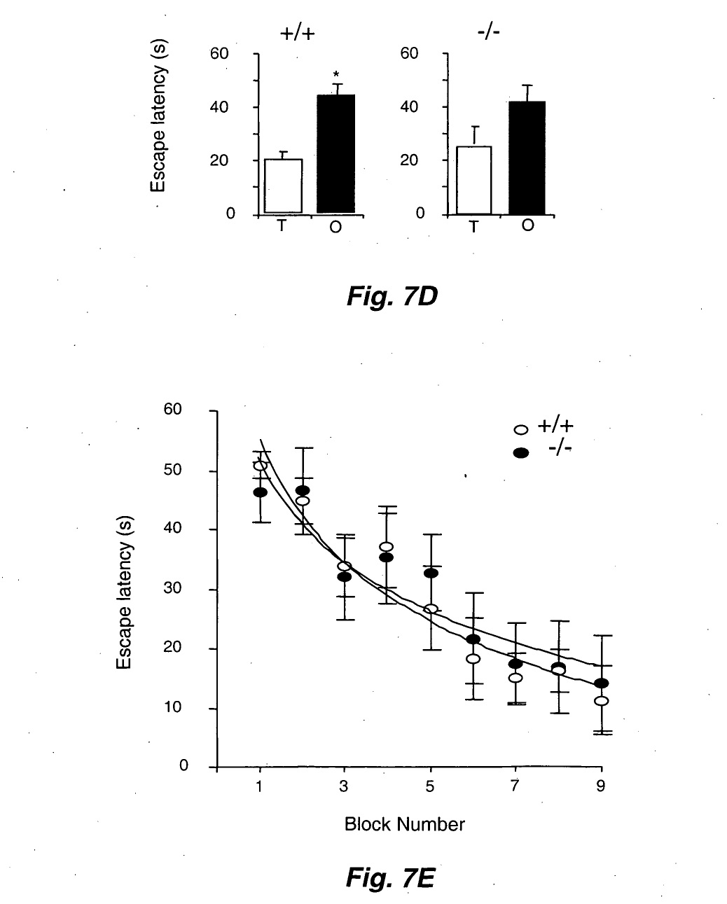

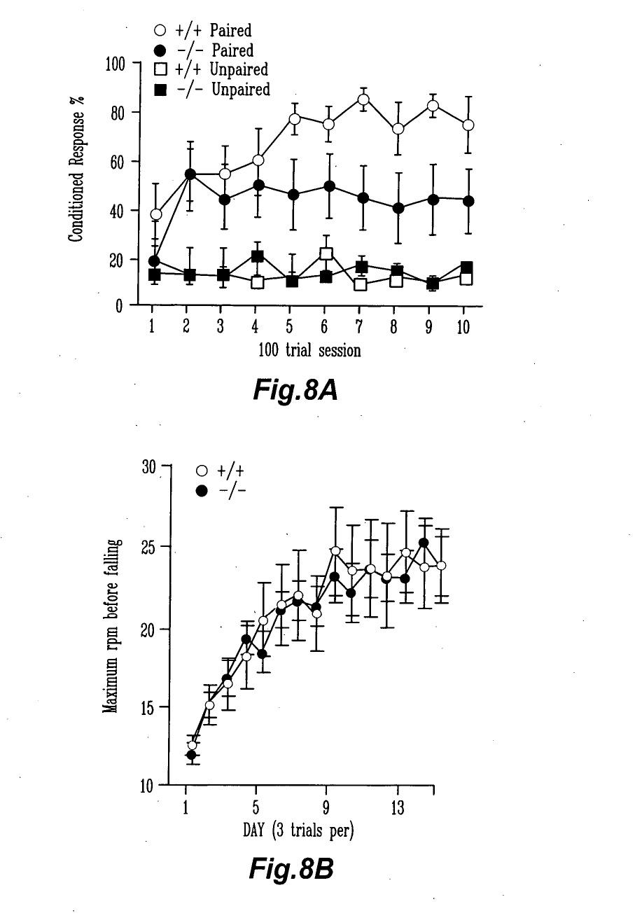

In this test, mice must learn the position of a submerged hidden platform relative to visual cues outside the pool. Naïve mice received a single trial per day for 11 consecutive days. Escape latencies of both +/+ and −/− mice improved significantly during the course of training (FIG. 7A). However, beyond day 3, the +/+ group was significantly faster at locating the platform that the −/− group. These results indicate that although the −/− mice could learn to find the location of the platform, their memory was less stable resulting in poorer retention from one training day to the next.

At the end of the training protocol, a probe trial was performed to examine whether mice had used spatial learning strategies to find the platform rather than other non-spatial strategies. The inventors found subtle differences in the performance of null mice during the probe trial (FIG. 7B, C). The +/+ mice spent a significantly greater amount of time in the training quadrant than in any of the other quadrants (FIG. 7B). In contrast, the amount of time ASIC −/− animals spent in the training quadrant was not significantly different from that spent in the other quadrants (FIG. 7B). An analysis of the number of platform crossings yielded similar results (FIG. 7C). Following the probe trial, a two trial platform reversal test was performed. In the first trial, the platform was returned to the original training quadrant. In the second trial, the platform was switched to the opposite quadrant. Wild type mice located the platform when it was in the training quadrant significantly faster than when it was in the opposite quadrant (FIG. 7D). In contrast, the times required for the knockout mice to locate the platform in the training and in the opposite quadrant were not statically different (FIG. 7D). Taken together, these results suggest that the ASIC −/− mice have a subtle deficit in spatial memory.

Our LTP experiments in hippocampal slices suggested that by amplifying NMDA receptor activation, the LTP deficits in the knockout mice could be rescued. Therefore the inventors tested whether an intensified training protocol could reverse the spatial learning deficit in the null mice. When the mice underwent 3 blocks of 4 trials per day for 3 consecutive days it was discovered that the performance of the +/+ and −/− groups were indistinguishable both in terms of escape latency (FIG. 7E) and probe trials (not shown). Thus, more intensive training reversed the ASIC dependent spatial learning deficit in the null mice.

### Example 8

### Loss of ASIC Impairs Eye-Blink Conditioning

Method of Eye-Blink Conditioning:

Eyeblink surgery. The +/+ (n=12) and −/− (n=12) mice were given i.p. injections of Nembutal® (1.6 ml/kg) and atropine sulfate (0.67 mg/kg) for anesthesia. They were then placed in a stereotaxic head holder and fitted with differential EMG electrodes that were implanted in the left eyelid muscle (orbicularis oculi). The EMG electrode leads terminated in gold pins in a plastic connector, which was secured to the skull with dental acrylic. A bipolar stimulating electrode (for delivering the shock US) was implanted subdermally, caudal to the left eye. The bipolar electrode terminated in a plastic connector that was secured to the skull by dental acrylic.
   Apparatus. The conditioning apparatus consisted of four small-animal sound attenuation chambers (BRS/LVE, Laurel, MD). Within each sound-attenuation chamber was a small-animal operant chamber (BRS/LVE, Laurel, MD) where the mice were kept during conditioning. One wall of the operant chamber was fitted with two speakers and a light. The electrode leads from the headstage were connected to peripheral equipment and a desktop computer. Computer software controlled the delivery of stimuli and the recording of eyelid EMG activity. EMG activity was recorded differentially, filtered and amplified.
Conditioning Procedure. The mice were assigned to either a paired or unpaired training condition, yielding four experimental groups: +/+ paired (n=6), −/− paired (n=6), +/+ unpaired (n=6), and −/− unpaired (n=6). In the paired condition, the mice were given 100 presentations of a tone conditioned stimulus (CS, 300 ms, 75 dB SPL, 2.0 kHz) and a shock unconditioned stimulus (US, 25 ms, 2.0 mA). The CS co-terminated with the US, yielding an interstimulus interval of 275 ms. Paired training trials were separated by a variable intertrial interval that averaged 30 s (range=18-42 s). In the unpaired condition, the mice were given explicitly unpaired presentations of the CS and US. The intertrial interval for unpaired training averaged 15 s (range=9-21 s). Conditioned responses (CRs) were defined as responses that crossed a threshold of 0.4 units (amplified and integrated units) above baseline during the CS period after 80 msec. Behavioral data were examined from digitized records of EMG responses.
Accelerating rotarod. After accommodation to the apparatus (Columbus Instruments, Columbus, Ohio), three trials per day were performed for 15 days. A trial consisted of 10 s at constant speed (3 rpm), followed by constant acceleration at 0.3 rpm per s until falling.

In addition to the hippocampus, ASIC transcripts are also expressed in granule and Purkinje cells in the cortex of the cerebellum (García-Añoveros et al., 1997; Waldmann et al., 1997b). Synapses between granule and Purkinje cells are likely sites for associative learning in classical eyeblink conditioning (Lavond et al., 1993; Mauk and Donegan, 1997; Thompson and Kim, 1996). Thus the inventors tested whether loss of ASIC could affect eyeblink conditioning. The basic procedure for eyeblink conditioning involves the paired presentation of an innocuous conditioned stimulus (CS) such as a tone, followed by a noxious unconditioned stimulus (US) such as a periorbital shock. With training, an association is made between the CS and the US so that a conditioned response (CR) is acquired. The coordinated motor response of the CR includes eyelid closure and is precisely timed to occur just prior to the delivery of the shock. Animals given unpaired presentations of CS and US do not develop the eyeblink CR, and thus serve as a control for non-associative sources of behavioral responses.

Although mice of both genotypes developed associative conditioning, the +/+ mice developed significantly stronger eyeblink conditioning than did the −/− mice (FIG. 8A). After 5 training sessions the tone generated a conditioned response approximately 80% of the time in wild type mice, whereas ASIC null mice showed a conditioned response of only about 50% of the time. The response percentage in the unpaired condition was not different between genotypes (FIG. 8A). Likewise, there was no significant difference in the amplitude of the unconditioned eyeblink response during the pre-training session. These results indicate that the impaired conditioning in the ASIC −/− mice was not due to a performance deficit. Thus as with spatial memory, the strength of eyeblink conditioning was impaired in the ASIC null mice.

To determine whether other cerebellum-dependent tasks were affected, inventors compared ASIC −/− and +/+ mice on the accelerating rotarod (FIG. 8B). The performance of the two groups was indistinguishable. Previously it has been shown that manipulations such as disrupting the glial fibrillary acidic protein (GFAP) or inhibition of PKC can affect cerebellar plasticity and eyeblink conditioning or the vestibulo-ocular reflex but do not lead to impaired performance on the rotating rod (De Zeeuw et al., 1998; Shibuki et al., 1996). Similar to these manipulations, the ASIC null mutation may affect specific forms of learning and plasticity.

Because ASIC is also expressed in sensory neurons (Chen et al., 1998; Waldmann et al., 1997a), a potential confounding factor in our behavioral studies could be a loss of peripheral sensory function (Price et al., 2000). However, the inventors tested mechanical and thermal sensation at the behavioral level and found no difference compared to littermate controls (not shown). This result agrees with the normal unconditioned eyeblink response (UR). In addition, the rotating rod provides a general test of coordination, strength, stamina, motivation, activity, and sensory function. The normal performance of the mutant mice in this task suggests that these characteristics are not grossly impaired.

Together these observations suggest that the observed differences in learning in the −/− mice are not likely the result of sensory or performance deficit.

Having described the invention with reference to particular compositions, theories of effectiveness, and the like, it will be apparent to those skilled in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary. It is to be further understood that all citations to articles, etc., herein are hereby expressly incorporated in their entirety by reference.

What is claimed is:

1. A pharmaceutical composition for treatment and prevention of strokes comprising:
an ASIC receptor antagonist and a pharmaceutically acceptable carrier.

2. A method for screening said composition as in claim 1 to identify said pharmaceutical which blocks a acid-sensing ion channels comprising:
administering the composition to be screened to cells,
expressing acid-gated channels in presence of acid and related peptides, and
determining whether the composition enhances or inhibits the opening of the acid-sensing ion channels of the DEG/ENaC channel family.

3. The method of claim 2 wherein the determination of opening of the acid-sensing ion channels is via electrophysical analysis.

4. The method of claim 3 wherein the electrophysical analysis looks for a current mediated by these channels.

5. The method of claim 3 wherein the electrophysical analysis looks for inactivation of a current in the channels.

6. The method of claim 2 wherein the determination of opening of the acid-sensing ion channels is via a method selected from the group consisting of voltage-sensitive dyes, ion-sensitive dyes, and cell death assays.

7. The method of claim 2 wherein the acid-gated channels are selected from the group consisting of ASICα, ASICβ and BNC1.

8. The method of claim 2 wherein the cells are selected from the group consisting of DRG neurons, Xenopus oocytes, cultured cell lines, and central nervous system cells.

9. A pharmaceutical composition for treatment and prevention of seizures comprising:
an ASIC receptor antagonist and a pharmaceutically acceptable carrier.

10. A method for screening said composition as in claim 9 to identify said pharmaceutical which blocks a acid-sensing ion channels comprising:
administering the composition to be screened to cells,
expressing acid-gated channels in presence of acid and related peptides, and
determining whether the composition enhances or inhibits the opening of the acid-sensing ion channels of the DEG/ENaC channel family.

11. The method of claim 10 wherein the determination of opening of the acid-sensing ion channels is via electrophysical analysis.

12. The method of claim 11 wherein the electrophysical analysis looks for a current mediated by these channels.

13. The method of claim 11 wherein the electrophysical analysis looks for inactivation of a current in the channels.

14. The method of claim 10 wherein the determination of opening of the acid-sensing ion channels is via a method selected from the group consisting of voltage-sensitive dyes, ion-sensitive dyes, and cell death assays.

15. The method of claim 10 wherein the acid-gated channels are selected from the group consisting of ASICα, ASICβ and BNC1.

16. The method of claim 10 wherein the cells are selected from the group consisting of DRG neurons, Xenopus oocytes, cultured cell lines, and central nervous system cells.

17. A pharmaceutical composition for treatment and prevention of memory loss comprising:
an ASIC receptor agonist and a pharmaceutically acceptable carrier.

18. A method for screening said composition to identify said pharmaceutical which activates a acid-sensing ion channels comprising:
administering the composition to be screened to cells,
expressing acid-gated channels in presence of acid and related peptides, and
determining whether the composition enhances or inhibits the opening of the acid-sensing ion channels of the DEG/ENaC channel family.

19. The method of claim 18 wherein the determination of opening of the acid-sensing ion channels is via electrophysical analysis.

20. The method of claim 19 wherein the electrophysical analysis looks for a current mediated by these channels.

21. The method of claim 19 wherein the electrophysical analysis looks for inactivation of a current in the channels.

22. The method of claim 18 wherein the determination of opening of the acid-sensing ion channels is via a method selected from the group consisting of voltage-sensitive dyes, ion-sensitive dyes, and cell death assays.

23. The method of claim 18 wherein the acid-gated channels are selected from the group consisting of ASICα, ASICβ and BNC1.

24. The method of claim 18 wherein the cells are selected from the group consisting of DRG neurons, Xenopus oocytes, cultured cell lines, and central nervous system cells.

25. A dietary supplement for treatment and prevention of strokes comprising:
an ASIC receptor antagonist and a pharmaceutically acceptable carrier.

26. A dietary supplement for treatment and prevention of seizures comprising:
an ASIC receptor antagonist and a pharmaceutically acceptable carrier.

27. A dietary supplement for treatment and prevention of memory loss comprising:
an ASIC receptor agonist and a pharmaceutically acceptable carrier.

28. A method of treating or preventing seizures comprising: administering a therapeutically effective amount of an ASIC antagonist.

29. A method according to claim 28 wherein the ASIC antagonist is contained in a pharmaceutically acceptable composition.

30. A method according to claim 28 wherein the pharmaceutically acceptable composition is administered by a route selected from the group consisting of orally, topically, sublingually, buccally, intranasally, rectally and intravenously.

31. A method of treating or preventing memory loss comprising:
administering a therapeutically effective amount of an ASIC agonist.

32. A method according to claim 31 wherein the ASIC agonist is contained in a pharmaceutically acceptable composition.

33. A method according to claim 31 wherein the pharmaceutically acceptable composition is administered by a route selected from the group consisting of orally, topically, sublingually, buccally, intranasally, rectally and intravenously.

34. A method of treating or preventing memory loss comprising:
administering a therapeutically effective amount of an ASIC agonist.

35. A method according to claim 34 wherein the ASIC agonist is contained in a pharmaceutically acceptable composition.

36. A method according to claim 34 wherein the pharmaceutically acceptable composition is administered by a route selected from the group consisting of orally, topically, sublingually, buccally, intranasally, rectally and intravenously.

37. A method for designing compositions which are an agonist, antagonist, or modulator of acid-sensing ion channels comprising:
determining the three-dimensional structure of the acid-sensing ion channels,
determining a composition which will bind with the channel, and
synthesizing the composition.

38. A composition as in claim 37 wherein said ASIC receptor antagonists exhibit modulation of excitatory neurotransmission.

39. A composition as in claim 37 wherein said ASIC receptor antagonists are inhibiting consequences of acidosis.

40. A composition as in claim 39 wherein said inhibition of acidosis effects the occurrence of seizures and strokes.

41. A composition as in claim 37 wherein said ASIC receptor antagonists have decreased adverse side effects on the patient.

42. A composition as in claim 37 wherein said ASIC receptor agonists are activating excitatory synaptic transmission.

43. A composition as in claim 42 wherein said activation of excitatory synaptic transmission effects learning and memory.

44. A method to treat a cognitive deficit linked to a neurological disorder comprising:
administering a therapeutically effective amount of a compound possessing functional antagonist properties for the acid-sensing ion receptor complex and a pharmaceutically acceptable carrier.

45. The method of claim 44 wherein the condition is selected from the group consisting of:
Alzheimer's, brain ischemia, cognitive disorder, affective disorders, diabetic ketoacidosis, diabetic retinopathy, excitotoxicity, Huntington's, hypoglycemia, kidney disease, memory deficiency neurologic disorder, and Parkinson's,

46. A method of reducing the effects of acidosis and excess glutamate release caused by strokes comprising:
inhibiting the function of an acid sensing ion channel.

47. The method of claim 46 wherein said inhibiting is by administering a therapeutically effective amount of an ASIC antagonist.

48. The method of claim 47 wherein said ASIC antagonist is contained in a pharmaceutically acceptable composition.

49. The method of claim 48 where the pharmaceutically acceptable composition is administered by a route selected from the group consisting of orally, topically, sublingually, buccally, intranasally, rectally, and intravenously.

50. A method of preventing cellular damage in a stroke patient comprising:
inhibiting or blocking the function of an acid sensing ion channel so that said channel is not activated by acidosis or excess glutamate present in the area of said stroke.
